SEQUENCE LISTING (52 sequences)

SEQ ID NO: 1 - DNA, 1182 bp, Bacillus subtilis
  CDS (1)..(1182)
atgtggttactcattatagcctgtgctgtcatactggtcatcggaatattagaaaaacgacgacatcagaaaaacattgatgccctccctgttcgggtgaatattaacggcatccgcggaaaatcgactgtgacaaggctgacaaccggaatattaatagaagccggttacaagactgttggaaaaacaacaggaacagatgcaagaatgatttactgggacacaccggaggaaaagccgattaaacggaaacctcaggggccgaatatcggagagcaaaaagaagtcatgagagaaacagtagaaagaggggctaacgcgattgtcagtgaatgcatggctgttaacccagattatcaaatcatctttcaggaagaacttctgcaggccaatatcggcgtcattgtgaatgttttggaagaccatatggatgtcatggggccgacgcttgatgaaattgcagaagcgtttactgctacaattccttataatggccatcttgtcattacagatagtgaatataccgagttctttaaacaaaaagcaaaagaacgaaacacaaaagtcatcattgctgataactcaaaaattacagatgagtatttacgtaaatttgaatacatggtattccctgataacgcttctctggcgctgggtgtggctcaagcactcggcattgacgaagaaacagcatttaagggaatgctgaatgcgccgccagatccgggagcaatgagaattcttccgctgatcagtccgagcgagcctgggcactttgttaatgggtttgccgcaaacgacgcttcttctactttgaatatatggaaacgtgtaaaagaaatcggttacccgaccgatgatccgatcatcatcatgaactgccgcgcagaccgtgtcgatcggacacagcaattcgcaaatgacgtattgccttatattgaagcaagtgaactgatcttaatcggtgaaacaacagaaccgatcgtaaaagcctacgaagaaggcaaaattcctgcagacaaactgcatgatctagagtataagtcaacagatgaaattatggaattgttaaagaaaagtatgcacaaccgtgtcatatatggcgtcggcaatattcatggtgccgcagagcctttaattgaaaaaatccacgaatacaaggtaaagcagctcgtaagctag

SEQ ID NO: 2 - protein, 393 aa, Bacillus subtilis
MWLLIIACAVILVIGILEKRRHQKNIDALPVRVNINGIRGKSTVTRLTTGILIEAGYKTVGKTTGTDARMIYWDTPEEKPIKRKPQGPNIGEQKEVMRETVERGANAIVSECMAVNPDYQIIFQEELLQANIGVIVNVLEDHMDVMGPTLDEIAEAFTATIPYNGHLVITDSEYTEFFKQKAKERNTKVIIADNSKITDEYLRKFEYMVFPDNASLALGVAQALGIDEETAFKGMLNAPPDPGAMRILPLISPSEPGHFVNGFAANDASSTLNIWKRVKEIGYPTDDPIIIMNCRADRVDRTQQFANDVLPYIEASELILIGETTEPIVKAYEEGKIPADKLHDLEYKSTDEIMELLKKSMHNRVIYGVGNIHGAAEPLIEKIHEYKVKQLVS

SEQ ID NO: 3 - DNA, 450 bp, Bacillus subtilis
  CDS (1)..(450)
atgttcggatcagatttatacatcgcactaattttaggtgtactactcagtttaatttttgcggaaaaaacagggatcgtgccggcaggacttgttgtaccgggatatttaggacttgtgtttaatcagccggtctttattttacttgttttgctagtgagcttgctcacgtatgtcattgtgaaatacggtttatccaaatttatgattttgtacggacgcagaaaattcgctgccatgctgataacagggatcgtcctaaaaatcgcgtttgattttctatacccgattgtaccatttgaaatcgcagaatttcgaggaatcggcatcatcgtgccaggtttaattgccaataccattcagaaacaaggtttaaccattacgttcggaagcacgctgctattgagcggagcgacctttgctatcatgtttgtttactacttaatttaa

SEQ ID NO: 4 - protein, 149 aa, Bacillus subtilis
MFGSDLYIALILGVLLSLIFAEKTGIVPAGLVVPGYLGLVFNQPVFILLVLLVSLLTYVIVKYGLSKFMILYGRRKFAAMLITGIVLKIAFDFLYPIVPFEIAEFRGIGIIVPGLIANTIQKQGLTITFGSTLLLSGATFAIMFVYYLI

SEQ ID NO: 5 - DNA, 1143 bp, Bacillus subtilis
  CDS (1)..(1143)
atgaaaaaagaactgagctttcatgaaaagctgctaaagctgacaaaacagcaaaaaaagaaaaccaataagcacgtatttattgccattccgatcgtttttgtccttatgttcgctttcatgtgggcgggaaaagcggaaacgccgaaggtcaaaacgtattctgacgacgtactctcagcctcatttgtaggcgatattatgatgggacgctatgttgaaaaagtaacggagcaaaaaggggcagacagtatttttcaatatgttgaaccgatctttagagcctcggattatgtagcaggaaactttgaaaacccggtaacctatcaaaagaattataaacaagcagataaagagattcatctgcagacgaataaggaatcagtgaaagtcttgaaggatatgaatttcacggttctcaacagcgccaacaaccacgcaatggattacggcgttcagggcatgaaagatacgcttggagaatttgcgaagcaaaatcttgatatcgttggagcgggatacagcttaagtgatgcgaaaaagaaaatttcgtaccagaaagtcaacggggtaacgattgcgacgcttggctttaccgatgtgtccgggaaaggtttcgcggctaaaaagaatacgccgggcgtgctgcccgcagatcctgaaatcttcatccctatgatttcagaagcgaaaaaacatgcggacattgttgttgtgcagtcacactggggacaagagtatgacaatgatccaaatgaccgccagcgccagcttgcaagagccatgtctgatgcgggagctgacatcatcgtcggccatcacccgcacgtcttagaaccgattgaagtatataacggaaccgtcattttctacagcctcggcaactttgtctttgaccaaggctggacgagaacaagagacagtgcactggttcagtatcacctgaagaaaaatggaacaggacgctttgaagtgacaccgatcgatatccatgaagcgacacctgcgcctgtgaaaaaagacagccttaaacagaaaaccattattcgcgaactgacgaaagactctaatttcgcttggaaagtagaagacggaaaactgacgtttgatattgatcatagtgacaaactaaaatctaaataa

SEQ ID NO: 6 - protein, 380 aa, Bacillus subtilis
MKKELSFHEKLLKLTKQQKKKTNKHVFIAIPIVFVLMFAFMWAGKAETPKVKTYSDDVLSASFVGDIMMGRYVEKVTEQKGADSIFQYVEPIFRASDYVAGNFENPVTYQKNYKQADKEIHLQTNKESVKVLKDMNFTVLNSANNHAMDYGVQGMKDTLGEFAKQNLDIVGAGYSLSDAKKKISYQKVNGVTIATLGFTDVSGKGFAAKKNTPGVLPADPEIFIPMISEAKKHADIVVVQSHWGQEYDNDPNDRQRQLARAMSDAGADIIVGHHPHVLEPIEVYNGTVIFYSLGNFVFDQGWTRTRDSALVQYHLKKNGTGRFEVTPIDIHEATPAPVKKDSLKQKTIIRELTKDSNFAWKVEDGKLTFDIDHSDKLKSK

SEQ ID NO: 7 - DNA, 1764 bp, Bacillus subtilis
  CDS (1)..(1764)
atgaaaagaacgtggaacgtctgtttaacagctctgcttagtgttctgttagtcgctggaagtgtcccttttcacgcggaagctaaaaaaccgcccaaaagctacgatgagtacaaacaagtagatgttggaaaagacggcatggttgcgaccgcacatcctcttgcttctgaaatcggtgctgatgtgctgaaaaaaggaggaaatgctattgacgcagcggttgccattcaatttgcactcaatgtaacagagccgatgatgtcaggtattggcggcggcggttttatgatggtgtatgacggaaaaacgaaggatacaacgataatcgacagccgtgagcgtgctccagcaggcgcaactcctgatatgtttctggacgaaaacggcaaagcaattcctttctctgaacgtgtaacaaaaggtactgccgttggtgttccaggcactctgaaagggctggaagaagccttggataaatggggaacccgttcgatgaagcaattaattaccccttctattaaactcgctgaaaaaggctttccgattgattcggtgttggcagaggccatttctgattatcaggaaaagctttcacggactgccgcaaaagatgtatttttaccaaatggcgaaccgcttaaagaaggagatacccttattcaaaaggatttggctaaaacatttaagcttattcgctccaaaggcactgacgctttttataaaggaaaattcgccaagacgctttctgacactgtccaggatttcggcggatcaatgacagaaaaagatttagaaaattacgacattacaattgatgaaccgatttggggagattatcaaggctatcaaatcgctactactcctcctccaagctccggcggtattttcttattgcaaatgctgaaaatccttgatcattttaacctttcacaatacgatgtccgctcatgggaaaaatatcagctgcttgctgaaacgatgcatttgtcatatgccgaccgtgcgtcttacgcaggtgatcccgaatttgtaaatgttcctctcaaaggcctgcttcaccccgattatattaaagaacgccagcaattaatcaacctagatcaagtgaataaaaaaccgaaagccggtgacccttggaaataccaagaaggatcagcaaactataaacaagttgaacagccgaaagacaaagtagaaggccaaacaacccactttacagttgctgaccgttggggaaatgttgtttcttatacaacaacaatcgaacagctattcggaacgggtattatggtccctgattacggtgttattttaaacaatgaattaacggattttgatgcgataccaggcggagctaacgaagtacagccaaacaaacggcctttaagcagcatgaccccgacgattttatttaaggatgacaagcctgtcctcacggttggatctcctggcggggccacaattatttcatccgttttgcaaaccattctctaccacattgaatatggtatggaattaaaagcagctgttgaagagccgagaatttacacaaacagcatgagctcttaccgttacgaagacggagttcctaaagatgtcctcagcaagctaaacggcatgggccacaaattcggcacaagtccggtggatatcggaaacgtgcaaagtatatcgatcgaccatgaaaacggcacctttaaaggtgtagctgattcaagcagaaacggcgcggcgatcggcattaatttaaaacgtaaataa

SEQ ID NO: 8 - protein, 587 aa, Bacillus subtilis
MKRTWNVCLTALLSVLLVAGSVPFHAEAKKPPKSYDEYKQVDVGKDGMVATAHPLASEIGADVLKKGGNAIDAAVAIQFALNVTEPMMSGIGGGGFMMVYDGKTKDTTIIDSRERAPAGATPDMFLDENGKAIPFSERVTKGTAVGVPGTLKGLEEALDKWGTRSMKQLITPSIKLAEKGFPIDSVLAEAISDYQEKLSRTAAKDVFLPNGEPLKEGDTLIQKDLAKTFKLIRSKGTDAFYKGKFAKTLSDTVQDFGGSMTEKDLENYDITIDEPIWGDYQGYQIATTPPPSSGGIFLLQMLKILDHFNLSQYDVRSWEKYQLLAETMHLSYADRASYAGDPEFVNVPLKGLLHPDYIKERQQLINLDQVNKKPKAGDPWKYQEGSANYKQVEQPKDKVEGQTTHFTVADRWGNVVSYTTTIEQLFGTGIMVPDYGVILNNELTDFDAIPGGANEVQPNKRPLSSMTPTILFKDDKPVLTVGSPGGATIISSVLQTILYHIEYGMELKAAVEEPRIYTNSMSSYRYEDGVPKDVLSKLNGMGHKFGTSPVDIGNVQSISIDHENGTFKGVADSSRNGAAIGINLKRK

SEQ ID NO: 9 - DNA, 575 bp, Bacillus subtilis
gatttgccgatgcaacaggcttatatttagaggaaatttctttttaaattgaatacggaataaaatcaggtaaacaggtcctgattttatttttttgagttttttagagaactgaagattgaaataaaagtagaagacaaaggacataagaaaattgcattagttttaattatagaaaacgcctttttataattatttatacctagaacgaaaatactgtttcgaaagcggtttactataaaaccttatattccggctcttttttaaaacagggggtaaaaattcactctagtattctaatttcaacatgctataataaatttgtaagacgcaatatgcatctctttttttacgatatatgtaagcggttaaccttgtgctatatgccgatttaggaaggggggtagattgagtcaagtagtaataatatagataacttataagttgttgagaagcaggagagcatctgggttactcacaagtttttttaaaactttaacgaaagcactttcggtaatgcttatgaatttagctatttgattcaattactttaaaaatatttaggaggtaatatg

SEQ ID NO: 10 - DNA, 567 bp, Bacillus subtilis
caaaagcagtccacacaaaacatgccccagcggcaatcgggccttattcacaagggattatcgtcaacaatatgttttacagctcaggccaaatccctttgactccttcaggcgaaatggtgaatggcgatattaaggagcagactcatcaagtattcagcaatttaaaggcggttctggaagaagcgggtgcttcttttgaaacagttgtaaaagcaactgtatttatcgcggatatggaacagtttgcggaagtaaacgaagtgtacggacaatattttgacactcacaaaccggcgagatcttgtgttgaagtcgcgagactcccgaaggatgcgttagtcgagatcgaagttattgcactggtgaaataataagaaaagtgattctgggagagccgggatcacttttttatttaccttatgcccgaaatgaaagctttatgacctaattgtgtaactatatcctattttttcaaaaaatattttaaaaacgagcaggatttcagaaaaaatcgtggaattgatacactaatgcttttatatagggaaaaggtggtgaactact

SEQ ID NO: 11 - DNA, 500 bp, Bacillus subtilis
tatttctcatcggtacgacaaactatcccgaagagatcgatccaggtttgatgaatcgtgcaggacgatttgaccgtgcctatgaaatcgggcttccggatgaagagctgcggctggaatatatgaaaatgagaggctttggcatctttttgagtgaaggagaaataaaaaacgccgcaaaacttacagaaggcttttcctttgcacagctgggagaattatatgtatcttcagcccttcaatggcaccaagaagggaatcaccatattgaaaccatggtgaaagacatgacaggagagcaaagaaaaagccagcggggaagctggatggaaagaaacaaagtcggttttcactaaaagaaagcacgggtgtttgaaaaacccgtgcttttttgttgcggttagccgaaattcgacaattgcggttattttgcgttcttctttttcttgtaaatatgataaaatatgacatatctcgggtaattcaaaaggggggattaa

SEQ ID NO: 12 - DNA, 40 bp, Artificial Sequence
  Oligonucleotide as PCR forward primer for amplifying pgsBCA region
atttaggaggtaatatgatgtggttactcattatagcctg

SEQ ID NO: 13 - DNA, 31 bp, Artificial Sequence
  Oligonucleotide as PCR reverse primer for amplifying pgsBCA region
cgaagcttagatggctttgacaaatttcatc

SEQ ID NO: 14 - DNA, 28 bp, Artificial Sequence
  Oligonucleotide as PCR forward primer for amplifying pgsBC region
cccaagcttgaccttcggcgtttccgct

SEQ ID NO: 15 - DNA, 30 bp, Artificial Sequence
  Oligonucleotide as PCR reverse primer for amplifying pgsBC region
cccaagcttggcagcgaattttctgcgtcc

SEQ ID NO: 16 - DNA, 44 bp, Artificial Sequence
  Oligonucleotide as PCR forward primer for amplifying a promoter region of S237 cellulase gene
caactaaagcacccattagggatccaacaggcttatatttagag

SEQ ID NO: 17 - DNA, 32 bp, Artificial Sequence
  Oligonucleotide as PCR reverse primer for amplifying a promoter region of S237 cellulase gene
catcatattacctcctaaatatttttaaagta

SEQ ID NO: 18 - DNA, 20 bp, Artificial Sequence
  Oligonucleotide as PCR forward primer for amplifying an upstream region of ggt gene
tccttcatgtctttcgtata

SEQ ID NO: 19 - DNA, 38 bp, Artificial Sequence
  Oligonucleotide as PCR reverse primer for amplifying an upstream region of ggt gene
ctaatgggtgctttagttggttctccctcctatatgaa

SEQ ID NO: 20 - DNA, 39 bp, Artificial Sequence
  Oligonucleotide as PCR forward primer for amplifying a downstream region of ggt gene
ctgccccgttagttgaagataaaaaactgtactcgcttc

SEQ ID NO: 21 - DNA, 21 bp, Artificial Sequence
  Oligonucleotide as PCR reverse primer for amplifying a downstream region of ggt gene
tagccaatatcacttttcatc

SEQ ID NO: 22 - DNA, 30 bp, Artificial Sequence
  Oligonucleotide as PCR forward primer for amplifying a region including chloramphenicol-resistant gene
caactaaagcacccattagttcaacaaacg

SEQ ID NO: 23 - DNA, 27 bp, Artificial Sequence
  Oligonucleotide as PCR reverse primer for amplifying a region including chloramphenicol-resistant gene
cttcaactaacggggcaggttagtgac

SEQ ID NO: 24 - DNA, 20 bp, Artificial Sequence
  Oligonucleotide as PCR forward primer for amplifying spoVG-UP fragment
atgaagtttcgtcgcagcgg

SEQ ID NO: 25 - DNA, 43 bp, Artificial Sequence
  Oligonucleotide as PCR reverse primer for amplifying spoVG-UP fragment
ctaatgggtgctttagttgtcatgattctgtctctccattctt

SEQ ID NO: 26 - DNA, 42 bp, Artificial Sequence
  Oligonucleotide as PCR forward primer for amplifying spoVG-DW fragment
ctgccccgttagttgaagcaaaagcagtccacacaaaacatg

SEQ ID NO: 27 - DNA, 28 bp, Artificial Sequence
  Oligonucleotide as PCR reverse primer for amplifying spoVG-DW fragment
catagtagttcaccaccttttccctata

SEQ ID NO: 28 - DNA, 42 bp, Artificial Sequence
  Oligonucleotide as PCR forward primer for amplifying comp_Cm fragment
caactaaagcacccattaggttagtgacattagaaaaccgac

SEQ ID NO: 29 - DNA, 45 bp, Artificial Sequence
  Oligonucleotide as PCR reverse primer for amplifying comp_Cm fragment
cttcaactaacggggcagttcaacaaacgaaaattggataaagtg

SEQ ID NO: 30 - DNA, 48 bp, Artificial Sequence
  Oligonucleotide as PCR forward primer for amplifying pgsB1 fragment
tatagggaaaaggtggtgaactactatgtggttactcattatagcctg

SEQ ID NO: 31 - DNA, 24 bp, Artificial Sequence
  Oligonucleotide as PCR reverse primer for amplifying pgsB1 fragment
gtctgcatttccccctagcttacg

SEQ ID NO: 32 - DNA, 37 bp, Artificial Sequence
  Oligonucleotide as PCR forward primer for amplifying pgsB-UP fragment
ctgccccgttagttgaagtgcttttcgacatctcctt

SEQ ID NO: 33 - DNA, 19 bp, Artificial Sequence
  Oligonucleotide as PCR reverse primer for amplifying pgsB-UP fragment
aagggtttgtgatatccgg

SEQ ID NO: 34 - DNA, 42 bp, Artificial Sequence
  Oligonucleotide as PCR reverse primer for amplifying P_spoVG/pgsBC fragment
ctgccccgggatccgaagcaaaagcagtccacacaaaacatg

SEQ ID NO: 35 - DNA, 20 bp, Artificial Sequence
  Oligonucleotide as PCR forward primer for amplifying rapA-UP fragment
tgaaaagatgcgtgcatttc

SEQ ID NO: 36 - DNA, 41 bp, Artificial Sequence
  Oligonucleotide as PCR reverse primer for amplifying rapA-UP fragment
ctaatgggtgctttagttgaatagcccctcttttgatgtcg

SEQ ID NO: 37 - DNA, 46 bp, Artificial Sequence
  Oligonucleotide as PCR forward primer for amplifying rapA-DW fragment
ctgccccgttagttgaagtatttctcatcggtacgacaaactatcc

SEQ ID NO: 38 - DNA, 23 bp, Artificial Sequence
  Oligonucleotide as PCR reverse primer for amplifying rapA-DW fragment
catttaatccccccttttgaatt

SEQ ID NO: 39 - DNA, 43 bp, Artificial Sequence
  Oligonucleotide as PCR forward primer for amplifying pgsB2 fragment
aattcaaaaggggggattaaatgtggttactcattatagcctg

SEQ ID NO: 40 - DNA, 46 bp, Artificial Sequence
  Oligonucleotide as PCR forward primer for amplifying P_rapA/pgsBC fragment
ctgccccgggatccgaagtatttctcatcggtacgacaaactatcc

SEQ ID NO: 41 - DNA, 21 bp, Artificial Sequence
  Oligonucleotide as PCR forward primer for amplifying tet-P_B fragment
catattgttgtataagtgatg

SEQ ID NO: 42 - DNA, 41 bp, Artificial Sequence
  Oligonucleotide as PCR forward primer for amplifying pgsB-DW1 fragment
catcacttatacaacaatatgtgcttttcgacatctccttc

SEQ ID NO: 43 - DNA, 41 bp, Artificial Sequence
  Oligonucleotide as PCR forward primer for amplifying Cm-PS237 fragment
taaatataagcctgttggatcccaactaaagcacccattag

SEQ ID NO: 44 - DNA, 27 bp, Artificial Sequence
  Oligonucleotide as PCR forward primer for amplifying a fragment of pgsBC gene
cccaagcttcttcaactaacggggcag

SEQ ID NO: 45 - DNA, 17 bp, Artificial Sequence
  Oligonucleotide as PCR forward primer for amplifying BCA-UP fragment
caagccccgagcaatca

SEQ ID NO: 46 - DNA, 38 bp, Artificial Sequence
  Oligonucleotide as PCR reverse primer for amplifying BCA-UP fragment
ctaatgggtgctttagttgcgaaagactctaatttcgc

SEQ ID NO: 47 - DNA, 1179 bp, Bacillus subtilis KSM-366
  CDS (1)..(1179)
atgtggttactcattatagcctgtgctgtcatactggtcatcggaatattagaaaaacgacgacatcagaaaaacattgatgccctccctgttcgggtgaatattaacggcatccgcggaaaatcgactgtgacaaggctgacaaccggaatattaatagaagccggttacaagactgttggaaaaacaacaggaacagatgcaagaatgatttactgggacacaccggaggaaaagccgattaaacggaaacctcaggggccgaatatcggagagcaaaaagaagtcatgagagaaacagtagaaagaggggctaacgcgattgtcagtgaatgcatggctgttaacccagattatcaaatcatctttcaggaagaacttctgcaggccaatatcggcgtcattgtgaatgttttagaagaccatatggatgtcatggggccgacgcttgatgaaattgcagaagcgtttaccgctacaattccttataatggccatcttgtcattacagatagtgaatataccgagttctttaaacaaaaagcaaaagaacgaaacacaaaagtcatcattgctgataactcaaaaattacagatgagtatttacgtaaatttgaatacatggtattccctgataacgcttctctggcgctgggtgtggctcaagcactcggcattgacgaagaaacagcatttaagggaatgctgaatgcgccgccagatccgggagcaatgagaattcttccgctgatcagtccgagcgagcctgggcactttgttaatgggtttgccgcaaacgacgcttcttctactttgaatatatggaaacgtgtaaaagaaatcggttacccgaccgatgatccgatcatcatcatgaactgccgcgcagaccgtgtcgatcggacacagcaattcgcaaatgacgtattgccttatattgaagcaagtgaactgatcttaatcggtgaaacaacagaaccgatcgtaaaagcctacgaagaaggcaaaattcctgcagacaaactgcatgatctagagtataagtcaacagatgaaattatggaattgttaaagaaaagaatgcacaaccgtgtcatatatggcgtcggcaatattcatggtgccgcagagcctttaattgaaaaaatccacgaatacaaggttaagcagctcgtaagc

SEQ ID NO: 48 - protein, 393 aa, Bacillus subtilis KSM-366
MWLLIIACAVILVIGILEKRRHQKNIDALPVRVNINGIRGKSTVTRLTTGILIEAGYKTVGKTTGTDARMIYWDTPEEKPIKRKPQGPNIGEQKEVMRETVERGANAIVSECMAVNPDYQIIFQEELLQANIGVIVNVLEDHMDVMGPTLDEIAEAFTATIPYNGHLVITDSEYTEFFKQKAKERNTKVIIADNSKITDEYLRKFEYMVFPDNASLALGVAQALGIDEETAFKGMLNAPPDPGAMRILPLISPSEPGHFVNGFAANDASSTLNIWKRVKEIGYPTDDPIIIMNCRADRVDRTQQFANDVLPYIEASELILIGETTEPIVKAYEEGKIPADKLHDLEYKSTDEIMELLKKRMHNRVIYGVGNIHGAAEPLIEKIHEYKVKQLVS

SEQ ID NO: 49 - DNA, 447 bp, Bacillus subtilis KSM-366
  CDS (1)..(447)
atgttcggatcagatttatacatcgcactaattttaggtgtactactcagtttaatttttgcggaaaaaacagggatcgtgccggcaggacttgttgtaccgggatatttaggacttgtgtttaatcagccggtctttattttacttgttttgctagtgagcttgctcacttatgttatcgtgaaatacggtttatccaaatttatgattttgtacggacgcagaaaattcgctgccatgctgataacagggatcgtcctaaaaatcgcgtttgattttctatacccgattgtaccatttgaaatcgcagaatttcgaggaatcggcatcatcgtgccaggtttaattgccaataccattcagaaacaaggtttaaccattacgttcggaagcacgctgctattgagcggagcgacctttgctatcatgtttgtttactacttaatt

SEQ ID NO: 50 - protein, 149 aa, Bacillus subtilis KSM-366; identical to SEQ ID NO: 4

SEQ ID NO: 51 - DNA, 1140 bp, Bacillus subtilis KSM-366
  CDS (1)..(1140)
atgaaaaaagaactgagctttcatgaaaagctgctaaagctgacaaaacagcaaaaaaagaaaaccaataagcacgtatttattgccattccgatcgtttttgtccttatgttcgctttcatgtgggcgggaaaagcggaaacgccgaaggtcaaaacgtattctgacgacgtactctcagcctcatttgtaggcgatattatgatgggacgctatgttgaaaaagtaacggagcaaaaaggggcagacagtatttttcaatatgttgaaccgatctttagagcctcggattatgtagcaggaaactttgaaaacccggtaacctatcaaaagaattataaacaagcagataaagagattcatctgcagacgaataaggaatcagtgaaagtcttgaaggatatgaatttcacggttctcaacagcgcaaacaaccacgcaatggattacggcgttcagggcatgaaagatacgcttggagaatttgcgaagcaaaaccttgatatcgttggagcgggatacagcttaagtgatgcgaaaaagaaaatttcgtaccagaaagtcaacggggtaacgattgcgacgcttggctttaccgatgtgtccgggaaaggtttcgcggctaaaaagaatacgccgggcgtgctgcccgcagatcctgaaatcttcatccctatgatttcagaagcgaaaaaacatgcggacatcgttgttgtgcagtcacactggggccaagagtatgacaatgatccaaacgaccgccagcgccagcttgcaagagccatgtctgatgcgggagctgacatcatcgtcggccatcacccgcacgtcttagaaccgattgaagtatataacggaaccgtcattttctacagcctcggcaactttgtctttgaccaaggctggacgagaacaagagacagtgcactggttcagtatcacctgaagaaaaatggaacaggccgctttgaagtgacaccgatcgacatccatgaagcgacacctgcgcctgtgaaaaaagacagccttaaacagaaaaccattattcgcgaactgacgaaagactctaatttcgcttggaaagtagaagacggaaaactgacgtttgatattgatcatagtgacaaactaaaatctaaa

SEQ ID NO: 52 - protein, 380 aa, Bacillus subtilis KSM-366; identical to SEQ ID NO: 6

(19) United States
(12) Patent Application Publication
(10) Pub. No.: US 2010/0248308 A1
(43) Pub. Date: Sep. 30, 2010

(54) Recombinant Microorganism and a Method for Producing Poly-Gamma-Glutamic Acid

(76) Inventors: Kazuhisa Sawada, Haga-gun (JP); Hiroshi Hagihara, Haga-gun (JP)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/678,798

(22) PCT Filed: Sep. 19, 2008
(86) PCT No.: PCT/JP2008/067023

§ 371 Date: Mar. 18, 2010

(30) Foreign Application Priority Data
Sep. 20, 2007 (JP) 2007-244023

(51) Int. Cl. C12P 21/02; C12N 1/21; C12N 15/75

(52) U.S. Cl. 435/69.1; 435/252.31; 435/471

(57) ABSTRACT

A recombinant microorganism having poly-γ-glutamic acid-producing ability, prepared by introducing a Bacillus subtilis pgsB gene or a gene functionally equivalent thereto, and a Bacillus subtilis pgsC gene or a gene functionally equivalent thereto, among a group of genes relating to synthesis of poly-γ-glutamic acid, into a host microorganism, in which no Bacillus subtilis pgsA gene or a gene functionally equivalent thereto is being introduced into the host microorganism; and a method of producing poly-γ-glutamic acid employing the recombinant microorganism.

### TECHNICAL FIELD

The present invention relates to a recombinant microorganism having poly-γ-glutamic acid-producing ability and a method of producing poly-γ-glutamic acid by employing the recombinant microorganism.

### BACKGROUND ART

Poly-γ-glutamic acid is a polymeric compound of glutamic acid, in which the γ-carboxyl and α-amino groups thereof are bound to each other, forming peptide bonds. The poly-γ-glutamic acid is also called γ-polyglutamic acid. The poly-γ-glutamic acid is known as a viscous substance produced by Bacillus subtilis var. natto. This compound has been attracting attention as a new polymeric raw material because of its various properties. In the description below, “poly-γ-glutamic acid” will be also referred to simply as “PGA”.

Examples of PGA-producing microorganisms include some of Bacillus microbes including Bacillus subtilis var. natto and the allied species (Bacillus subtilis var. chungkookjang, Bacillus licheniformis, Bacillus megaterium, Bacillus anthracis, and Bacillus halodurans), Natrialba aegyptiaca, and Hydra (see, for example, Non-Patent Document 1). Among the microorganisms above, the PGA-producing Bacillus subtilis var. natto is taxonomically classified into a subspecies of PGA-nonproducing Bacillus subtilis, and it is known that any strain of the microorganism has pgsB, pgsC and pgsA genes coding PGA-producing enzymes at sites downstream of the same transcription initiation regulatory region and the same translation initiation regulatory region, forming a cluster structure (operon) (see, for example, Non-Patent Documents 3, 6 and 8). In addition, the PGA production-controlling mechanism of Bacillus subtilis var. natto is considered to be regulated in a complicated way in which various intercellular control factors are involved, similarly to the mechanism of Bacillus subtilis acquiring transforming ability (see, for example, Non-Patent Documents 9 and 10). It is possible to improve the productivity of the PGA by breeding of the PGA-producing microorganism as a method of producing PGA. However, such a method demands a great amount of labor needed for elimination of the strict regulation mechanism in wild strain, elimination of complicated nutritional requirements in wild strain, or optimized stabilized productivity of the production process. Thus, it is not considered to be an efficient method for producing PGA. For that reason, use of gene recombination technology is studied as an efficient method replacing the conventional breeding of wild strains. For example, as a method of producing PGA by the gene recombination technology, it was disclosed that a Bacillus subtilis recombinant containing genes introduced with plasmid (Bacillus subtilis ISW1214 strain) has a productivity of approximately 9.0 g/L/5 days (see Non-Patent Document 2) and that a Escherichia coli recombinant containing genes introduced with plasmid has a productivity of approximately 4 g/L/1.5 days (see Non-Patent Document 7) or of 2.5 mg/40 mg-drying microbe (see Non-Patent Document 6). However, the productivity by these producing methods disclosed above is still considered insufficient.

PGA is shown to be ribosome-independently produced by a membrane-binding enzyme system called PgsBCA in Bacillus subtilis var. natto (see, for example, Non-Patent Document 4). Among the enzymes in PGA synthetase system PgsBCA, PgsB (which is also referred to as YwsC) is known to be involved in amide ligase reaction, as it acts on the γ-carboxyl group of glutamic acid (see, e.g., Non-Patent Documents 3 and 5). On the other hand, according to Non-Patent Document 3, PgsC (which is also referred to as YwtA) is considered to be a membrane-binding protein involved in extracellular excretion of PGA. However, according to Non-Patent Documents 4 and 5. PgsC is said to be involved in ligation reaction of glutamic acids in combination with PgsB. In this situation, the function of PgsC remains unresolved even now.

Further, Non-Patent Document 2 discloses the results obtained by studying the PGA productivity of recombinant Bacillus subtilis strains that are deficient of the pgsB, pgsC and pgsA genes on chromosome but contain an introduced vector having one gene selected from pgsB, pgsC and pgsA genes or a vector having multiple genes selected therefrom in combination. Non-Patent Document 2 concludes that expression of all pgsB, pgsC and pgsA genes are needed for production of PGA. Alternatively, in Non-Patent Document 3, a strain deficient in pgsB gene (which is also referred to as ywsC gene), a strain deficient in pgsC gene (which is also referred to as ywtA gene) and a strain deficient in pgsA gene (which is also referred to as ywtB gene) of Bacillus subtilis var. natto (IFO16449 strain) having an ability of producing PGA were prepared and the ability of producing PGA of the deficient strains was examined. Non-Patent Document 3 concludes that the pgsB gene and the pgsC gene are essential in PGA production in Bacillus subtilis var. natto having an ability of producing PGA and the pgsA gene is needed for further improvement in productivity of PGA.

Patent Document 1 also discloses that Escherichia coli strain inherently having no ability of producing PGA gains the ability of producing PGA, when transformed with a vector containing pgsB gene, pgsC gene and pgsA gene in that order. Non-Patent Document 6 discloses that, in a recombinant Escherichia coli strain, PGA was produced under the condition when a pgsBCA gene was introduced, but no PGA was produced under the condition when a pgsBC gene was introduced.

Patent Documents 2 to 5 disclose that PGAs having an average molecular weight of several thousands to 2,000,000 were obtained when a PGA-producing microorganism such as Bacillus subtilis var. natto was cultured in various nutrition salt media. Patent Document 6 discloses that a strain of Bacillus subtilis var. natto produced PGA having a molecular weight of 3,000,000 or more under application of external stress.

Non-Patent Document 11 and Patent Documents 7 and 8 disclosed that it is possible to produce PGA high in optical purity by microorganisms. Patent Document 7 discloses that it is possible to produce PGA having a L-glutamic acid content of 90% or more at a productivity of approximately 10 g/L/5 days by employing a Bacillus megaterium strain. Patent Document 8 discloses that a Natrialba aegyptiaca mutant strain produces poly-γ-L-glutamic acid having a molecular weight of 1,300,000 or more at a productivity of approximately 5 g/L/6 days.

[Non-Patent Document 1] Ashiuchi, M., et al.: Appl. Microbiol. Biotechnol., 59, pp. 9-14 (2002)
[Non-Patent Document 2] Ashiuchi, M., et al.: Biosci. Biotechnol. Biochem., 70, pp. 1794-1797 (2006)

### [Non-Patent Document 3] Urushibata, Y., et al.: J. Bacteriol., 184, pp. 337-343 (2002)

### [Non-Patent Document 4] Makoto ASHIUCHI, et al.: Mirai Zairyo (Materials for the Future), 3, pp. 44-50 (2003)

[Non-Patent Document 5] Ashiuchi, M., et al.: Eur. J. Biochem., 268, pp. 5321-5328 (2001)
[Non-Patent Document 6] Ashiuchi, M., et al.: Biochem. Biophys. Res. Commun., 263, pp. 6-12 (1999)
[Non-Patent Document 7] Jiang, H., et al.: Biotechnol. Lett., 28, pp. 1241-1246 (2006)

### [Non-Patent Document 8] Presecan, E., et al.: Microbiology, 143, pp. 3313-3328 (1997)

[Non-Patent Document 9] Itaya, M., et al.: Biosci. Biotechnol. Biochem., 63, pp. 2034-2037 (1999)
[Non-Patent Document 10] Tadayuki IMANAKA, et al.: Biseibutsu Riyou no Daitenkai (Great Development of Use of Microorganisms), first chapter, fourth section, pp. 657-663 (2002)
[Non-Patent Document 11] Shimizu, K., et al.: Appl. Environ. Microbiol., 73, pp. 2378-2379 (2007)
[Patent Document 1] JP-A-2001-17182 (“JP-A” means unexamined published Japanese patent application)
[Patent Document 2] JP-B-43-24472 (“JP-B” means examined Japanese patent publication)

### [Patent Document 3] JP-A-1-174397

### [Patent Document 4] JP-A-3-47087

### [Patent Document 5] Japanese Patent No. 3081901

### [Patent Document 6] JP-A-2006-42617

### [Patent Document 7] JP-A-2007-228957

### [Patent Document 8] JP-A-2007-314434

### DISCLOSURE OF INVENTION

However, no method of producing PGA at high yield was disclosed in Non-Patent Documents 1 to 3 and 7 and Patent Document 1 described above, and unfavorably, it was impossible to produce PGA efficiently by traditional methods. There was also no report on adjustment of the molecular weight of PGA by genetic engineering method.

The present invention, which was made under the circumstances above, resides in to provide: a recombinant microorganism capable of producing PGA efficiency; a method of producing PGA by employing the microorganism; a method of improving PGA productivity by introduction of genes; a method of adjusting molecular weight of PGA; and a method of adjusting optical purity of PGA.

After intensive studies, the inventors found that it is possible to produce PGA in high productivity, to adjust molecular weight of PGA produced, and/or to adjust optical purity of PGA produced, by employing a recombinant microorganism obtained by introducing a pgsB gene or a gene functionally equivalent thereto and a pgsC gene or a gene functionally equivalent thereto into a host microorganism. The present invention was made, based on the finding above.

The recombinant microorganism according to the present invention is a microorganism obtained by introducing a Bacillus subtilis pgsB gene or a gene functionally equivalent thereto and a Bacillus subtilis pgsC gene or a gene functionally equivalent thereto, among a group of genes relating to synthesis of PGA (pgsBCA genes), into a host microorganism. However, a Bacillus subtilis pgsA gene or a gene functionally equivalent thereto is not being introduced into the host microorganism. Thus, the present invention is made, based on the finding that the PGA productivity is rather better when the Bacillus subtilis pgsA gene or a gene functionally equivalent thereto is not being introduced, in contrast with traditional understanding.

The pgsB gene is preferably a gene coding the following protein (a) or (b).

(a) Protein having the amino acid sequence set forth in SEQ ID NO: 2
(b) Protein having an amino acid sequence, in which one or more amino acids are being deleted, substituted, added or inserted in the amino acid sequence set forth in SEQ ID NO: 2, and having amido-ligase activity

The pgsC gene is preferably a gene coding the following protein (c) or (d).

(c) Protein having the amino acid sequence set forth in SEQ ID NO: 4
(d) Protein having an amino acid sequence, in which one or more amino acids are being deleted, substituted, added or inserted in the amino acid sequence set forth in SEQ ID NO: 4, and having a function of producing PGA in the present of the PgsB protein coded by the pgsB gene.

In addition, in the recombinant microorganism according to the present invention, it is preferably that the pgsB gene or the gene functionally equivalent thereto and the pgsC gene or the gene functionally equivalent thereto are bonded to the downstream of a transcription initiation regulatory region and/or a translation initiation regulatory region functioning in the host cell, the expression of which is positively regulated.

The host microorganism for the recombinant microorganism according to the present invention is preferably a Bacillus microbe. An examples of the Bacillus microbe includes Bacillus subtilis.

The method of producing PGA according to the present invention is a method of culturing the recombinant microorganism according to the present invention described above and collecting the PGA produced.

The method of improving the productivity of PGA according to the present invention is a method characterized by improving PGA productivity by employing a recombinant microorganism obtained by introducing a Bacillus subtilis pgsB gene or a gene functionally equivalent thereto and a Bacillus subtilis pgsC gene or a gene functionally equivalent thereto, among a group of genes relating to synthesis of PGA, into a host microorganism.

The method of adjusting molecular weight of PGA according to the present invention is a method characterized by adjusting the molecular weight of PGA (preferably making the PGA have high molecular) by employing a recombinant microorganism obtained by introducing a Bacillus subtilis pgsB gene or a gene functionally equivalent thereto and a Bacillus subtilis pgsC gene or a gene functionally equivalent thereto, among a group of the genes relating to synthesis of PGA, into a host microorganism.

The method of adjusting optical purity of PGA according to the present invention is a method characterized by adjusting the optical purity of PGA (preferably increasing L-isomer ratio of PGA) by employing a recombinant microorganism obtained by introducing a Bacillus subtilis pgsB gene or a gene functionally equivalent thereto and a Bacillus subtilis pgsC gene or a gene functionally equivalent thereto, among a group of genes relating to synthesis of PGA, into a host microorganism.

### BRIEF DESCRIPTION OF DRAWINGS

FIG. 1 is a flow chart for construction of a recombinant vector for producing PGA, for showing an example of the procedure of the steps of cloning the genes involved in PGA synthesis.

FIG. 2 is a flow chart showing the procedure for deleting a target gene through double crossing method by using the SOE-PCR fragments obtained in Preparation Example 5.

FIG. 3 is a flow chart for construction of a recombinant vector for producing PGA, for showing the procedure of the steps of cloning the genes involved in PGA synthesis in Preparation Examples 2 and 3.

FIG. 4 is a flow chart for construction of a recombinant vector for producing PGA, for showing the procedure of the steps of cloning the genes involved in PGA synthesis in Preparation Example 4.

Other and further features and advantages of the invention will appear more fully from the following description, appropriately referring to the accompanying drawings.

### BEST MODE FOR CARRYING OUT THE INVENTION

Hereinafter, the present invention will be described in detail.

The recombinant microorganism according to the present invention is a microorganism obtained by introducing a Bacillus subtilis pgsB gene or a gene functionally equivalent thereto and a Bacillus subtilis pgsC gene or a gene functionally equivalent thereto, among a group of genes relating to synthesis of PGA, into a host microorganism, but not introducing a Bacillus subtilis pgsA gene into the host microorganism. The recombinant microorganism has ability of producing poly-γ-glutamic acid.

Herein, the group of genes relating to synthesis of PGA include the pgsB gene (BG12531: also called ywsC gene or capB gene), the pgsC gene (BG12532: also called ywtA gene or capC gene) and the pgsA gene (BG12533: also called ywtB gene or capA gene) in Bacillus subtilis. Bacillus subtilis Marburg No. 168 strain, which is widely used as a host microorganism for gene recombinant, has these pgsB gene, pgsC gene and pgsA gene on its chromosome, but does not have ability of producing PGA. Similarly, Bacillus subtilis var. natto has these pgsB gene, pgsC gene and pgsA gene on its chromosome (genome) and has ability of producing PGA. The name and the number of each gene are described, according to the Bacillus subtilis genome data disclosed on Internet (http://bacillus.genome.ad.jpi, updated on Jan. 18) by JAFAN [Japan Functional Analysis Network for Bacillus subtilis (BSORF DB)].

The nucleotide sequence of the pgsB gene is set forth in SEQ ID NO: 1, and the amino acid sequence of the PgsB protein coded by the pgsB gene is set forth in SEQ ID NO: 2. In the present invention, the pgsB gene is not limited to the gene having the nucleotide sequence set forth in SEQ ID NO: 1, and examples thereof include a gene coding a protein having a modified amino acid sequence of SEQ ID NO: 2 in which one or more amino acids are being deleted, substituted, added or inserted and having amido-ligase activity by using glutamic acid as substrate. The number of amino acids modified then may be, for example, 2 to 80, preferably 2 to 40, more preferably 2 to 20, still more preferably 2 to 10, and most preferably 2 to 5.

In the present invention, the pgsB gene includes a gene having a nucleotide sequence with a genetic identity to the nucleotide sequence set forth in SEQ ID NO: 1 of 80% or more, preferably 90% or more, more preferably 95% or more, more preferably 96% or more, still more preferably 97% or more, most preferably 98% or more, and particularly preferably 99% or more; and coding a protein having amido-ligase activity. The homology of the nucleotide sequence is calculated through the Lipman-Pearson method (see Lipman, D J., Pearson, W R.: Science, 227, pp. 1435-1441 (1985)). Specifically, the homology can be determined through use of a homology analysis (Search homology) program of genetic information processing software Genetyx-Win (Software Development Co., Ltd.) (Unit size to compare (ktup)=2). When a reaction solution containing glutamic acid and ATP as substrates and additionally manganese chloride is used, the amido-ligase activity can be determined by measuring the PGA generated in the reaction solution [see, for example, Urushibata. Y., et al.: J. Bacteriol., 184, pp. 337-343 (2002)].

In the present invention, the pgsB gene includes a gene capable of hybridizing with a complementary sequence to the nucleotide sequence set forth in SEQ ID NO: 1 under a stringent condition, and coding a protein having amido-ligase activity. The “stringent condition” above is, for example, the condition described in “Molecular Cloning—A LABORATORY MANUAL THIRD EDITION” [Joseph Sambrook, David W. Russell, Cold Spring Harbor Laboratory Press (2001)]. It is, for example, a hybridization condition of a gene with a probe by incubation thereof in a solution containing 6×SSC (1×SSC composition: 0.15 M sodium chloride and 0.015 M sodium citrate, pH 7.0), 0.5% SDS, 5×Denhardt and 100 mg/mL herring sperm DNA at 65° C. for 8 to 16 hours.

The amido-ligase activity described above is an activity to produce PGA extracellularly when the pgsB gene is introduced together with the pgsC gene into a host microorganism, as will be described below in Examples in detail. The ability of producing PGA of a particular microorganism can be determined, for example, by a method of visually observing the semitransparent viscous substance formed around a colony when the microorganism is cultured on a PGA production agar medium containing glutamic acid or the salt thereof, a method of detecting PGA by SDS-PAGE [see, for example, Yamaguchi, F., et al.: Biosci. Biotechnol. Biochem., 60, pp. 255-258 (1996)], a method of quantitatively determining PGA after acid hydrolysis by using an amino acid analyzer [see, for example, Ogawa, Y., et al.: Biosci. Biotechnol. Biochem., 61, pp. 1684-1687 (1997)], a quantitative method of determining the dry weight after purification of the culture solution [see, for example, Ashiuchi, M., et al.: Biochem. Biophys. Res. Commun., 263: pp. 6-12 (1999)], or a quantitative/qualitative method by HPLC (high-performance liquid chromatography) by using a gel filtration column [see, for example, Kubota, H., et al.: Biosci. Biotechnol. Biochem., 57, pp. 1212-1213 (1993)].

The nucleotide sequence of the pgsC gene is set forth in SEQ ID NO: 3, and the amino acid sequence of the PgsC protein coded by the pgsC gene is set forth in SEQ ID NO: 4. In the present invention, the pgsC gene is not limited to the gene having the nucleotide sequence set forth in SEQ ID NO: 3, and examples thereof includes a gene coding a protein having a modified amino acid sequence of SEQ ID NO: 4 in which one or more amino acids are being deleted, substituted, added or inserted and having ability of producing PGA in the presence of the PgsB protein described above. The number of amino acids modified then may be, for example, 2 to 30, preferably 2 to 15, more preferably 2 to 8, still more preferably 2 to 2, and most preferably 2.

In the present invention, the pgsC gene includes a gene having a nucleotide sequence having a genetic identity to the nucleotide sequence set forth in SEQ ID NO: 3 of 80% or more, preferably 90% or more, more preferably 95% or more, more preferably 96% or more, still more preferably 97% or more, most preferably 98% or more and particularly preferably 99% or more; and coding a protein having ability of producing PGA in the presence of the PgsB protein described above. The identity of nucleotide sequence is the same as that defined above.

In the present invention, the pgsC gene includes a gene capable of hybridizing with a complementary sequence to the nucleotide sequence set forth in SEQ ID NO: 3 under a stringent condition, and coding a protein having ability of producing PGA in the presence of the PgsB protein described above. The “stringent condition” is also the same as that defined above.

The ability of the PgsC protein of producing PGA in the presence of the PgsB protein described above means to produce PGA extracellularly when the pgsC gene is expressed with the pgsB gene in a host microorganism. As described in detail below in Examples and Reference Example, the present invention is based on the finding by the present inventors that a host microorganism expressing the pgsB gene alone does not produce PGA, while that expressing both the pgsB gene and pgsC gene do produce PGA.

The recombinant microorganism according to the present invention is not limited to the above-described host microorganism containing the introduced pgsB and pgsC genes derived from Bacillus subtilis, and may include, for example, host microorganisms having an introduced gene functionally equivalent to the pgsB gene (pgsB homologous gene) and an introduced gene functionally equivalent to the pgsC gene (pgsC homologous gene), among a group of genes relating to synthesis of PGA derived from microorganisms other than Bacillus subtilis. The pgsB homologous gene and the pgsC homologous gene can be isolated and identified from a known microorganism capable of producing PGA by a common method. Examples of the microorganism having the ability of producing PGA include, in addition to Bacillus subtilis, Bacillus licheniformis, Bacillus megaterium, Bacillus anthracis, Bacillus halodurans, Natrialba aegyptiaca, and Hydra. The pgsB homologous gene and the pgsC homologous gene can be isolated and identified from these microorganisms. The pgsB homologous gene can be isolated, for example, by extracting genomic DNA from any of the microorganisms and performing Southern hybridization while the polynucleotide having the nucleotide sequence set forth in SEQ ID NO: 1 above is used as probe. The pgsC homologous gene can also be isolated similarly. Further, in recent rapid progress in genome sequencing, the Bacillus subtilis pgsB homologous gene as defined above can be isolated and identified even in a microorganism incapable of producing PGA, based on the genome sequence information. The Bacillus subtilis pgsC homologous gene can be isolated in the same manner as described above.

The recombinant microorganism according to the present invention is prepared by introducing the pgsB gene or the pgsB homologous gene described above and the pgsC gene or the pgsC homologous gene into a host microorganism.

The host microorganism for use in the present invention is not particularly limited, and typical examples thereof include Bacillus microbes such as Bacillus licheniformis, Bacillus megaterium, Bacillus anthracis, Bacillus halodurans, and Bacillus subtilis; Clostridium microbes, Corynebacterium microbes, Escherichia coli, and yeasts, and Bacillus microbes are preferable. In particular, Bacillus subtilis is preferable, because the entire genome information is known, the genetic and genomic engineering technologies thereof are established, and there are available many microbial strains that are established as safe and favorable host microorganisms.

The host cell to be chosen may be a wild-type cell or a mutant-type strain containing a particular mutation introduced into the wild-type cell. In particular, among the mutant strains, it would be possible to obtain favorable effect, by employing a mutant strain, in which a gene coding a enzyme for decomposing PGA (a gene of enzyme for decomposing PGA) is being deleted, as the host microorganism. A known gene of enzyme for decomposing PGA in Bacillus subtilis is a ggt gene [see, for example, Kimura, K., et al.: Microbiology, 150, pp. 4115-4123 (2003)]. The nucleotide sequence of the ggt gene is set forth in SEQ ID NO: 7, and the amino acid sequence of the enzyme for decomposing PGA coded by the ggt gene is set forth in SEQ ID NO: 8. When a microorganism other than Bacillus subtilis is used as the host, use of a mutant strain with the ggt-equivalent gene deleted is preferable. In the present invention, the ggt gene is not limited to the gene having the nucleotide sequence set forth in SEQ ID NO: 7, and examples thereof include a gene coding a protein having a modified amino acid sequence of SEQ ID NO: 8 in which one or more amino acids are being deleted, substituted, added or inserted and having activity of decomposing PGA. The number of amino acids modified then may be, for example, 2 to 120, preferably 2 to 60, more preferably 2 to 30, still more preferably 2 to 15, and most preferably 2 to 8. In the present invention, the ggt gene includes a gene having a nucleotide sequence having a genetic identity to the nucleotide sequence set forth in SEQ ID NO: 7 of 80% or more, preferably 90% or more, more preferably 95% or more, more preferably 96% or more, still more preferably 97% or more, most preferably 98% or more and particularly preferably 99% or more; and coding a protein having activity of decomposing PGA. The identity of nucleotide sequence is the same as that defined above.

In the present invention, the ggt gene includes a gene capable of hybridizing with a complementary sequence to the nucleotide sequence set forth in SEQ ID NO: 8 under a stringent condition, and coding a protein having activity of decomposing PGA. The “stringent condition” is also the same as that defined above. The ggt gene or the homologous gene thereof can be deleted properly by using a known method. For example, it is possible to introducing a cyclic recombinant plasmid, which is obtained by cloning a DNA fragment containing a part of the ggt gene into a suitable plasmid (vector), into a host microorganism cell and inactivates the target gene on the host microorganism genome by fragmenting it by homologous mutation in a partial region of the target gene [see Leenhouts, K., et al.: Mol. Gen. Genet., 253, pp. 217-224 (1996)].

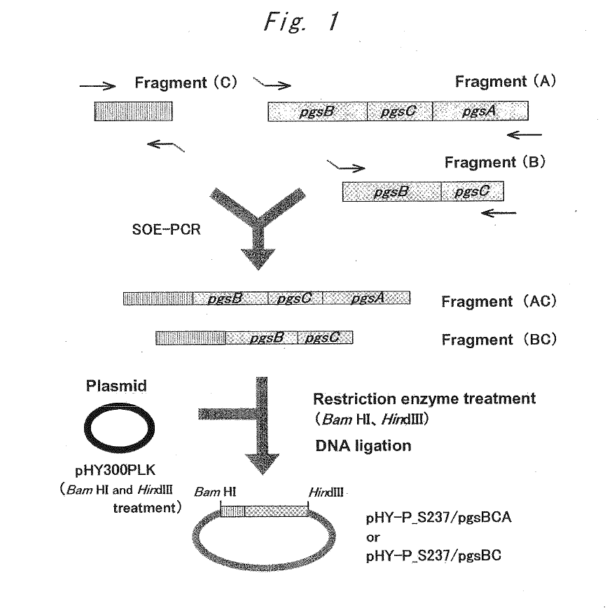

In particular, when Bacillus subtilis is used as the host microorganism for construction of the microorganism according to the present invention, there are some reported methods of deleting or inactivating the target gene by homologous recombination [see, for example, Itaya, M., Tanaka, T.: Mol. Gen. Genet., 223, pp. 268-272 (1990)], and it is possible to obtain the desired host microorganism by repeating these methods. Inactivation of random genes can also be performed by use of a mutagenetic agent or by irradiation on host microorganism with ultraviolet ray, γ-ray or the like. In such a case, it is also possible to obtain similar effect, for example, by mutagenesis by means of base substitution of nucleotides or insertion of nucleotides, or deletion of part of the gene, by similar mutagenesis in the regulatory regions such as promoter region, or by similar mutagenesis of the genes dependent on the expression control of the ggt gene. It is also possible to delete the ggt gene on genome by a more efficient method of constructing a straight-chain DNA fragment containing the upstream and downstream regions of the ggt gene but not containing the ggt gene by SOE (splicing by overlap extension)-PCR method [see, for example, Horton. R M., et al.: Gene, 77, pp. 61-68 (1989)], introducing it into a host microorganism cell, and thus, causing homologous recombination by double crossing at two sites outside the ggt gene on the host microorganism genome (see FIG. 1).

The host cell chosen may be a microorganism inherently having ability of producing PGA or a microorganism inherently having no ability of producing PGA. It may be thus a microorganism having the pgsBCA gene on chromosome (genome) and having ability of producing PGA, a microorganism having the pgsBCA gene on chromosome but no ability of producing PGA, or a microorganism having no pgsBCA gene at all. Introduction of the pgsB gene or the pgsB homologous gene and the pgsC gene or the pgsC homologous gene described above into a microorganism having ability of producing PGA leads to increase in ability of producing PGA and/or in the molecular weight of PGA produced. On the other hand, introduction of the pgsB gene or the pgsB homologous gene and the pgsC gene or the pgsC homologous gene described above into a microorganism having no ability of producing PGA leads to addition of the ability of producing PGA.

It is possible to use, as the host cell, a microorganism having ability of producing PGA which is obtained by introducing therein a transcription initiation regulatory region and/or a translation initiation regulatory region functioning in the microorganism, at the sites upstream of the pgsBCA gene or a gene functionally equivalent thereto of Bacillus subtilis on chromosome (genome).

The transcription initiation regulatory region and/or the translation initiation regulatory region functioning in the host microorganism according to the present invention (hereinafter, referred to as “regulatory region”) are preferably bound thereto in a suitable form, and the transcription initiation regulatory region and the translation initiation regulatory region are preferably bound to each other.

The regulatory region is more preferably a region that can constitutively express downstream genes bound to the region and increase the expression amount of the downstream genes in host cell. Examples of the regulatory region include a regulatory region of an α-amylase gene, a regulatory region of a protease gene, a regulatory region of an aprE gene, a regulatory region of a spoVG gene and a regulatory region of a rapA gene derived from Bacillus microbes; a regulatory region of a cellulase gene derived from Bacillus sp. KSM-S237 strain; and a regulatory region of a kanamycin resistance gene and a regulatory region of a chloramphenicol resistance gene derived from Staphylococcus aureus.

In introduction of the regulatory region on the genome upstream of the pgsBCA gene or the gene functionally equivalent thereto, part or all of the inherent regulatory region for the pgsBCA gene or the gene functionally equivalent thereto of Bacillus subtilis is used for substitution.

The substitution to the regulatory region may be performed by any known method, for example, by homologous recombination (e.g., the method described in Mol. Gen. Genet., 223, 268, 1990).

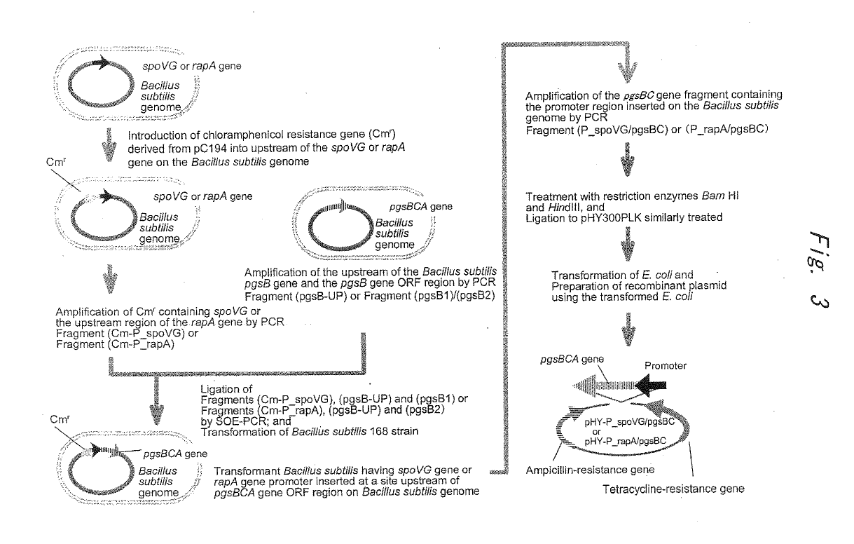

For example, first, a DNA fragment containing the upstream region of the transcription initiation regulatory region inherent to the pgsBCA gene and a drug-resistance gene fragment are bound to the sites upstream of the DNA fragment containing the regulatory region, and a DNA fragment containing part or all of the translation initiation regulatory region of the pgsBCA gene or part or all of the structural gene region of the pgsBCA gene is bound to the site downstream the DNA fragment containing the regulatory region, by a common method such as SOE-PCR method. In this way, obtained is a DNA fragment containing a DNA fragment having the upstream region of the inherent transcription initiation regulatory region of the pgsBCA gene, a drug-resistance gene fragment, a DNA fragment having the regulatory region, and part or all of the translation initiation regulatory region of the pgsBCA gene and the structural gene region, and part or all of the structural gene region of the pgsBCA gene, in that order (see FIG. 3).

Subsequent incorporation of the DNA fragment into host microorganism cell by a common method results in homologous double-crossing recombination at two sites with the upstream region of the inherent transcription initiation regulatory region of the pgsBCA gene on the host microorganism genome, and part or all of the translation initiation regulatory region of the pgsBCA gene and the structural gene region or part or all of the structural gene region of the pgsBCA gene. As a result, the transformant having its inherent regulatory regions substituted with the regulatory regions above can be separated by employing the drug-resistance gene as indicator. The regulatory region introduced to the genome upstream of the pgsBCA gene is thus retained as it is genetically stabilized.

The recombinant microorganism according to the present invention does not contain the pgsA gene introduced, among a group of the genes relating to synthesis of PGA in the host microorganism. The nucleotide sequence of the pgsA gene is set forth in SEQ ID NO: 5, while the amino acid sequence of the protein coded by the pgsA gene is set forth in SEQ ID NO: 6. The protein coded by the pgsA gene is suggested to be a protein involved in extracellular release of PGA (see, for example, the Non-Patent Document 5). However, the function thereof remains unresolved even now. Characteristically, the recombinant microorganism according to the present invention is superior in PGA productivity, even though it contains no pgsA gene introduced. The pgsA gene in the present invention is not limited to the gene having the nucleotide sequence set forth in SEQ ID NO: 5, and examples thereof includes a gene coding a protein having a modified amino acid sequence of SEQ ID NO: 6 in which one or more amino acids are being deleted, substituted, added or inserted, in which the protein has been considered to have PGA synthesis activity together with the PgsB and the PgsC. The number of amino acids modified then may be, for example, 2 to 80, preferably 2 to 40, more preferably 2 to 20, still more preferably 2 to 10, and most preferably 2 to 5. In the present invention, the pgsA gene includes a gene having a nucleotide sequence having an identity to the nucleotide sequence set forth in SEQ ID NO: 5 of 80% or more, preferably 90% or more, more preferably 95% or more, more preferably 96% or more, still more preferably 97% or more, most preferably 98% or more and particularly preferably 99% or more, and coding a protein having been considered to have PGA synthesis activity together with the PgsB and the PgsC. The identity of nucleotide sequence is the same as that defined above.

In the present invention, the pgsA gene includes a gene capable of hybridizing with a complementary sequence to the nucleotide sequence set forth in SEQ ID NO: 5 under a stringent condition, and coding a protein having been considered to have PGA synthesis activity together with the PgsB and the PgsC. The “stringent condition” is also the same as that defined above.

The phrase “no pgsA gene is being introduced into the host microorganism” refers to that no gene coding the PgsA protein functioning in the PGA synthesis process is introduced. Accordingly in the case where a gene having a higher identity with the gene set forth in SEQ ID NO: 5 is introduced, when the gene is not expressed or the protein produced by expression of the gene does not function in the PGA synthesis process, such a microorganism is also included in the technical scope of the present invention.

Preferably, in the recombinant microorganism according to the present invention, the pgsB gene or the pgsB homologous gene and the pgsC gene or the pgsC homologous gene described above have a transcription initiation regulatory region and a translation initiation regulatory region functioning in the host cell and positively expression-controlled that are bound to the sites upstream of the genes. The regulatory regions are more preferably regions expressing the downstream bound genes and increasing the expression amounts of the genes in host cell, and those expressing the downstream gene constitutively or/and in a greater amount are particularly preferable. Examples of the regulatory regions include, but are not limited to, a regulatory region of an α-amylase gene, a regulatory region of a protease gene, a regulatory region of an aprE gene, a regulatory region of a spoVG gene and a regulatory region of a rapA gene derived from Bacillus microbes; a regulatory region of a cellulase gene of Bacillus sp. KSM-S237 strain; and a regulatory region of a kanamycin resistance gene and a regulatory region of a chloramphenicol resistance gene derived from Staphylococcus aureus.

The nucleotide sequence of the regulatory region of the cellulase gene of the Bacillus sp. KSM-S237 strain, i.e., the regulatory region upstream by 0.4 to 1.0 kb of the translation initiation point of the cellulase gene, described above, is set forth in SEQ ID NO: 9. The regulatory region is not limited to that having the nucleotide sequence set forth in SEQ ID NO: 9, and it includes, for example, a nucleotide sequence having a genetic identity to the nucleotide sequence set forth in SEQ ID NO: 9 of 70% or more, preferably 80% or more, more preferably 90% or more, more preferably 96% or more, still more preferably 97% or more, most preferably 98% or more and particularly preferably 99% or more. The identity of nucleotide sequence is the same as that defined above.

The nucleotide sequence of the regulatory region of the spoVG gene (BG10112) derived from Bacillus subtilis is set forth in SEQ ID NO: 10. The regulatory region is not limited to that having the nucleotide sequence set forth in SEQ ID NO: 10, and it includes, for example, a nucleotide sequence having a genetic identity to the nucleotide sequence set forth in SEQ ID NO: 10 of 70% or more, preferably 80% or more, more preferably 90% or more, more preferably 96% or more, still more preferably 97% or more, most preferably 98% or more and particularly preferably 99% or more. The identity of nucleotide sequence is the same as that defined above.

The nucleotide sequence of the regulatory region of the rapA gene (BG10652) derived from Bacillus subtilis is set forth in SEQ ID NO: 11. The regulatory region is not limited to that having the nucleotide sequence set forth in SEQ ID NO: 11, and it includes, for example, a nucleotide sequence having a genetic identity to the nucleotide sequence set forth in SEQ ID NO: 11 of 70% or more, preferably 80% or more, more preferably 90% or more, more preferably 96% or more, still more preferably 97% or more, most preferably 98% or more and particularly preferably 99% or more. The identity of nucleotide sequence is the same as that defined above.

A common plasmid (vector) may be used in introducing the pgsB gene or the pgsB homologous gene and the pgsC gene or the pgsC homologous gene described above into a host microorganism. The plasmid may be selected properly according to the kind of the host microorganism to which the genes are to be introduced. Examples thereof, when Bacillus subtilis is the host, include pT181, pC194, pUB110, pE194, pSN2, and pHY300PLK. The plasmid to be selected is preferably a plasmid self-replicable in the host cell, and more preferably, there are multiple copies of the plasmid contained in cell. The copy number of plasmids with respect to the host genome (chromosome) is favorably 2 or more and 100 or less, preferably 2 or more and 50 or less, and more preferably 2 or more and 30 or less. A common method such as protoplast method [see, for example, Chamg, S., Cohen, S H.: Mol. Gen. Genet., 168, pp. 111-115 (1979)] and competent cell method [see, for example, Young, F E., Spizizen, J.: J. Bacteriol., 86, pp. 392-400 (1963)] may be used in transformation with the expression vector.

It is possible to produce PGA at excellent productivity by employing the recombinant microorganism according to the present invention described above. The PGA produced can be used in various applications such as cosmetics, medicines, foods, water purifiers, moisturizing materials, thickeners, and others. In particular, the recombinant microorganism according to the present invention, which improves PGA productivity significantly, compared to that by conventional genetic engineering technologies, allows drastic reduction of the production cost for PGA that is used in the applications above.

In particular, in production of PGA by employing the recombinant microorganism according to the present invention, the recombinant microorganism is first cultured in a suitable medium and the PGA extracellularly produced is collected. The medium for use is, for example, a medium in the composition containing carbon sources such as glycerol, glucose, fructose, maltose, xylose, arabinose, and various organic acids, nitrogen sources such as various amino acids, polypeptone, tryptone, ammonium sulfate, and urea, inorganic salts such as sodium salt and other needed nutrient sources, trace amounts of metal salts, and the like. The medium may be a synthetic medium or a natural medium.

In particular, for improvement in PGA productivity, it is preferable to use a medium containing glutamic acid in an amount of more than that needed for growth of the recombinant microorganism to be cultured. Specifically, it is preferable to add it, as sodium glutamate, to the medium, and the concentration therein is, for example, 0.005 to 600 g/L, preferably 0.05 to 500 g/L, more preferably 0.1 to 400 g/L, and most preferably 0.5 to 300 g/L. An excessively low sodium glutamate concentration in the medium may lead to complete consumption of the glutamic acid in the medium by the recombinant microorganism and consequently to insufficient improvement in PGA productivity. Alternatively, an excessively high glutamic acid concentration in the medium may lead to a problem of precipitation of sodium glutamate and other medium components if it is higher than the saturation solubility of sodium glutamate.

For collection of the PGA accumulated in the medium, it is necessary to remove the microorganism microbe that has produced PGA, for example, by means of centrifugation, ultrafiltration membrane, electrodialysis, isoelectric focusing of pH to the isoelectric point of PGA, or the like, and these methods may be used in combination.

In this way, it is possible to produce PGA by employing the recombinant microorganism according to the present invention. As will be described in Examples below, the recombinant microorganism according to the present invention shows a high productivity of 1.5 g/L or more, and even the recombinant microorganism having the introduced Bacillus subtilis pgsB gene or gene functionally equivalent thereto and Bacillus subtilis pgsC gene or gene functionally equivalent thereto but having no Bacillus subtilis pgsA gene or gene functionally equivalent thereto shows favorable PGA productivity, compared to microorganisms capable of producing PGA obtained by conventional genetic engineering technology. Thus, the recombinant microorganism according to the present invention is useful in industrial production of PGA.

As will be described in Example below, introduction of the Bacillus subtilis pgsB gene or gene functionally equivalent thereto and the Bacillus subtilis pgsC gene or gene functionally equivalent thereto into a microorganism having ability of producing PGA results in making the PGA produced have high molecular weight. Thus, introduction of these genes into the microorganism with ability of producing PGA enables regulation of the molecular weight of PGA produced, and moreover, use of the recombinant microorganism obtained enables efficient production of PGA having a desired molecular weight.

In particular, when a microorganism having the regulatory region of the cellulase gene derived from Bacillus sp. KSM-S237 strain bound to the upstream regulatory region of the pgsBCA gene is used, it is possible to produce a high-molecular-weight PGA having a weight-averaged molecular weight of approximately 7,000,000 to 7,800,000. Introduction of the Bacillus subtilis pgsB gene or gene functionally equivalent thereto and the Bacillus subtilis pgsC gene or gene functionally equivalent thereto to this microorganism allows control of the molecular weight of PGA, i.e., increase in PGA molecular weight preferably to 110 to 115%.

When a microorganism having the regulatory region of the rapA gene bound to the upstream regulatory region of the pgsBCA gene is used as the host cell, it is possible to produce PGA having medium-molecular weight, e.g., a weight-averaged molecular weight of approximately 590,000 to 600,000. When a microorganism having the introduced Bacillus subtilis pgsB gene or gene functionally equivalent thereto and the introduced Bacillus subtilis pgsC gene or gene functionally equivalent thereto is used, it is possible to adjust the molecular weight of PGA, i.e., increase in the molecular weight of PGA produced preferably to 1,200 to 1,600%.

Further, introduction of the Bacillus subtilis pgsB gene or gene functionally equivalent thereto and the Bacillus subtilis pgsC gene or gene functionally equivalent thereto into a microorganism having no ability of producing PGA enables control of the L-glutamic acid content to preferably 80% or more, more preferably 90% or more. In addition, according to the present invention, it is possible to produce PGA high in optical purity (e.g., at an optical purity of preferably 80 to 100%, more preferably 90 to 100%).

By employing the recombinant microorganism according to the present invention, it is possible to produce PGA having a molecular weight varying properly according to applications, and to reduce the production cost for the PGA used in various applications drastically, and to produce PGA high in optical purity. Thus, the recombinant microorganism is useful in industrial production of PGA.

### EXAMPLES

Hereinafter, the present invention will be described more in detail with reference to Examples, but it should be understood that the technological scope of the present invention is not particularly limited by the following Examples. The name, the sequence and the SEQ ID NO: of each of the primers used in Examples are shown in Table 1. The underlined region in the nucleotide sequence shown in Table 1 indicates the sequence of the restriction site added.

TABLE 1

| | | SEQ |
|---|---|---|
| | | ID |
| Primer | Nucleotide sequence | NO: |
| pgsB-F | ATTTAGGAGGTAATATGATGTGGTTACTCATTATA | 12 |
| | GCCTG | |
| pgsA-R | CGAAGCTTAGATGGCTTTGACAAATTTCATC | 13 |
| pgsC-R | CCCAAGCTTGACCTTCGGCGTTTCCGCT | 14 |
| pgsB-R | CCCAAGCTTGGCAGCGAATTTTCTGCGTCC | 15 |
| P_S237-FW | CAACTAAAGCACCCATTAGGGATCCAACAGGCTT | 16 |
| | ATATTTAGAG | |
| P S237-RV | CATCATATTACCTCCTAAATATTTTTAAAGTA | 17 |
| ggt-F | TCCTTCATGTCTTTCGTATA | 18 |
| ggt/Cm-R | CTAATGGGTGCTTTAGTTGGTTCTCCCTCCTATATG | 19 |
| | AA | |
| ggt/Cm-F | CTGCCCCGTTAGTTGAAGATAAAAAACTGTACTCG | 20 |
| | CTTC | |
| ggt-R | TAGCCAATATCACTTTTCATC | 21 |
| CmFW | CAACTAAAGCACCCATTAGTTCAACAAACG | 22 |
| CmRV | CTTCAACTAACGGGGCAGGTTAGTGAC | 23 |
| P-spoVG FW2 | ATGAAGTTTCGTCGCAGCGG | 24 |
| P-spoVG/Cm | CTAATGGGTGCTTTAGTTGTCATGATTCTGTCTCTC | 25 |
| R | CATTCTT | |
| P_spoVG/Cm | CTGCCCCGTTAGTTGAAGCAAAAGCAGTCCACAC | 26 |
| F | AAAACATG | |
| P_spoVG RV | CATAGTAGTTCACCACCTTTTCCCTATA | 27 |
| comp_Cm FW | CAACTAAAGCACCCATTAGGTTAGTGACATTAGA | 28 |
| | AAACCGAC | |
| comp_Cm RV | CTTCAACTAACGGGGCAGTTCAACAAACGAAAAT | 29 |
| | TGGATAAAGTG | |
| comp_P- | TATAGGGAAAAGGTGGTGAACTACTATGTGGTTA | 30 |
| spoVG | CTCATTATAGCCTG | |
| R/BSpgsB | | |
| atg FW | | |
| pgsC FW | GTCTGCATTTCCCCCTAGCTTACG | 31 |
| pgsB/Cm F | CTGCCCCGTTAGTTGAAGTGCTTTTCGACATCTCC | 32 |
| | TT | |
| pgsB RV | AAGGGTTTGTGATATCCGG | 33 |
| BamHI_ | CTGCCCCGGGATCCGAAGCAAAAGCAGTCCACAC | 34 |
| PspoVG FW | AAAACATG | |
| rapA FW | TGAAAAGATGCGTGCATTTC | 35 |
| P_rapA/Cm R | CTAATGGGTGCTTTAGTTGAATAGCCCCTCTTTTG | 36 |
| | ATGTCG | |
| P_rapA/Cm F | CTGCCCCGTTAGTTGAAGTATTTCTCATCGGTACG | 37 |
| | ACAAACTATCC | |
| P_rapA RV | CATTTAATCCCCCCTTTTGAATT | 38 |
| comp_P-rapA | AATTCAAAAGGGGGGATTAAATGTGGTTACTCATT | 39 |
| R/BSpgsB | ATAGCCTG | |
| atg FW | | |
| BamHI_PrapA | CTGCCCCGGGATCCGAAGTATTTCTCATCGGTACG | 40 |
| FW | ACAAACTATCC | |

### Preparation Example 1

### Construction of Vector (1)

A method of cloning genes relating to synthesis of PGA is shown in this Preparation Example (see FIG. 1).

A chromosome DNA, serving as a template, prepared from Bacillus sp. KSM-366 strain (FERM BP-6262) and a primer set of pgsB-F (SEQ ID NO: 12) and pgsA-R (SEQ ID NO: 13) shown in Table 1 were used to prepare a 2.9 kb DNA fragment (A) of pgsBCA gene. The nucleotide sequence of the pgsB gene of the Bacillus sp. KSM-366 strain is set forth in SEQ ID NO: 47, the amino acid sequence of the protein coded by the pgsB gene is set forth in SEQ ID NO: 48, the nucleotide sequence of the pgsC gene is set forth in SEQ ID NO: 49, the amino acid sequence of the protein coded by the pgsC gene is set forth in SEQ ID NO: 50, the nucleotide sequence of the pgsA gene is set forth in SEQ ID NO: 51, and the amino acid sequence of the protein coded by the pgsA gene is set forth in SEQ ID NO: 52. Separately, a primer set of pgsB-F and pgsC-R (SEQ ID NO: 14) shown in Table 1 was used to prepare a 1.7 kb DNA fragment (B) of pgsBC gene. Yet separately, a chromosome DNA, serving as a template, prepared from Bacillus sp. KSM-S237 strain (FERM BP-7875) and a primer set of P_S237-FW (SEQ ID NO: 16) and P_S237-RV (SEQ ID NO: 17) shown in Table 1 were used to prepare a 0.6 kb DNA fragment (C) of the promoter region of the cellulase gene derived from KSM-S237 strain (see JP-A-2000-210081).

Then, a 3.5 kb composite DNA fragment (AC) containing the fragments (A) and (C) was prepared by using the thus-prepared fragments (A) and (C) as templates and a primer set of P_S237-FW and pgsA-R by SOE-PCR method. Similarly, a 2.3 kb composite DNA fragment (BC) containing the fragments (B) and (C) was obtained by using the fragments (B) and (C) as templates and a primer set of P_S237-FW and pgsC-R by SOE-PCR method. These DNA fragments (AC) and (BC) were treated with restriction enzymes BamHI and HindIII, respectively, and ligated with (bound to) a plasmid vector pHY300PLK (Takara) previously treated with the same restriction enzymes by using DNA Ligation kit Ver. 2 (Takara).

The thus-prepared ligation sample was used to transform Escherichia coli (HB101 strain) by a competent cell method and cultured on, and a colony formed on a LB agar medium (LBTc agar medium) containing a 15-ppm tetracycline hydrochloride salt (SIGMA-ALDRICH) was collected as a transformant. The transformant obtained was grown once again on the LBTc agar medium, and recombinant plasmids were prepared from the obtained microbe by using High Pure Plasmid Isolation Kit (Roche Diagnostics). The recombinant plasmids were treated with the restriction enzymes BamHI and HindIII, and insertion of a desirable DNA fragment (AC) or (BC) into the BamHI and HindIII restriction sites of the plasmid vectors was confirmed by electrophoresis.

In this Preparation Example, recombinant plasmids confirmed to contain the DNA fragments (AC) and (BC) were designated recombinant vectors for producing PGA pHY-P_S237/pgsBCA and pHY-P_S237/pgsBC, respectively. The PCR reaction was carried out by using GeneAmp 9700 (PE Applied Biosystems) and TaKaRa LA Taq (Takara) according to the protocols attached to the kits.

### Preparation Example 2

### Construction of Vector (2)

A method of cloning genes relating to synthesis of is shown in this Preparation Example (see FIG. 3).

A chromosome DNA, serving as a template, prepared from Bacillus subtilis Marburg No. 168 stain (Bacillus subtilis 168 strain) and a primer set of P_spoVG FW2 (SEQ ID NO: 24) and P_spoVG/Cm R (SEQ ID NO: 25) shown in Table 1 were used to prepare an upstream 0.9 kb DNA fragment (spoVG-UP) for insertion of the chloramphenicol-resistant gene (Cm^{r}) derived from the plasmid pC194 [see, for example, Horinouchi, S., Weisblum, B.: J. Bacteriol., 150, pp. 815-825 (1982)] to a site approximately 0.6 kb upstream of spoVG gene ORF. Similarly, a primer set of P_spoVG/Cm F (SEQ ID NO: 26) and P_spoVG RV (SEQ ID NO: 27) shown in Table 1 was used to prepare a downstream 0.6 kb DNA fragment (spoVG-DW) (then, the primer P_spoVG RV was designed to modify the initiation codon of the spoVG gene ORF from gtg to atg). Separately, the plasmid pC194, serving as a template, and a primer set comp_Cm FW (SEQ ID NO: 28) and comp_Cm RV (SEQ ID NO: 29) shown in Table 1 were used to prepare a chloramphenicol-resistant gene 0.9 kb DNA fragment (comp_Cm).

Subsequently, the thus-prepared fragments (spoVG-UP), (spoVG-DW) and (comp_Cm), as templates, and a primer set of P_spoVG FW2 and P_spoVG RV were used to prepare a composite 2.4 kb DNA fragment (Cm-P_spoVG) of the three fragments by SOE-PCR method. The thus-prepared DNA fragment was used to transform a Bacillus subtilis 168 strain by the competent cell method (described above), and a colony growing on a LB agar medium (LBCm agar medium) containing 10 ppm of chloramphenicol (SIGMA_ALDRICH) was separated as a transformant.

Then, a genome DNA extracted from the thus-prepared transformant, serving as a template, and a primer set of comp_Cm FW and P_spoVG RV shown in Table 1 were used to prepare a 1.5 kb DNA fragment (P-spoVG-Cm2) containing the chloramphenicol-resistant gene and a promoter region of the spoVG gene by PCR. Separately, a primer set of comp_P-spoVG R/BSpgsB atg FW (SEQ ID NO: 30) and pgsC FW (SEQ ID NO: 31) and a genome DNA sample prepared from Bacillus subtilis 168 strain, as a template, were used to prepare a pgsB gene 1.2 kb DNA fragment (pgsB1) by PCR; and a primer set of pgsB/Cm F (SEQ ID NO: 32) and pgsB RV (SEQ ID NO: 33) and the template were used to prepare an upstream 1.1 kb DNA fragment (pgsB-UP) of pgsB gene ORF. Further, the thus-prepared three fragments (pgsB1), (P-spoVG-Cm2) and (pgsB-UP), as templates, and a primer set of pgsC FW and pgsB RV were used to prepare a 3.6 kb DNA fragment by SOE-PCR.

Subsequently, the thus-prepared DNA fragment was used to transform Bacillus subtilis 168 strain by the competent cell method, and a colony growing on a LBCm agar medium was separated as a transformant (hereinafter, the thus-obtained recombinant Bacillus subtilis is referred to as 1 cp-P_spoVG/pgsBCAcm strain). The genome DNA sample extracted from the transformant obtained, as a template, and a primer set of pgsC-R and BamHI_PspoVG FW (SEQ ID NO: 34) were used to prepare a 2.3 kb DNA fragment (P_spoVG/pgsBC) having a Bacillus subtilis spoVG gene promoter upstream of the Bacillus subtilis pgsBC gene. In addition, the thus-prepared DNA fragment was cloned into a plasmid vector in the same manner as in the Preparation Example 1, and the recombinant vector for producing PGA obtained was designated as pHY-P_spoVG/pgsBC.

### Preparation Example 3

### Construction of Vector (3)

In this Preparation Example, a method of cloning genes relating to synthesis of PGA is described, in a similar manner to the Preparation Example 2 (see FIG. 3).

A chromosome DNA, serving as a template, prepared from Bacillus subtilis 168 strain and a primer set of rapA FW (SEQ ID NO: 35) and P_rapA/Cm R (SEQ ID NO: 36) shown in Table 1 were used to prepare an upstream 0.5 kb DNA fragment (rapA-DW) for insertion of the chloramphenicol-resistant gene derived from the plasmid pC194 to a site approximately 0.5 kb upstream of rapA gene ORF. Similarly, a primer set of P_rapA/Cm F (SEQ ID NO: 37) and P_rapA RV (SEQ ID NO: 38) shown in Table 1 was used to prepare a downstream 0.5 kb DNA fragment (rapA-DW) (then, the primer P_rapA RV was designed to modify the initiation codon of the rapA gene ORF from ttg to atg).

Subsequently, the thus-prepared fragments (rapA-UP) and (rapA-DW), and the DNA fragment (comp_Cm) prepared in the Preparation Example 2, as templates, and a primer set of rapA FW and P_rapA RV were used to prepare a composite 1.9 kb DNA fragment (Cm-P_rapA) of the three fragments by SOE-PCR method. The thus-prepared DNA fragment was used to transform Bacillus subtilis 168 strain by the competent cell method, and a colony growing on a LBCm agar medium was separated as a transformant.

Then, a genome DNA extracted from the thus-prepared transformant, serving as a template, and a primer set of comp_Cm FW and P_rapA RV shown in Table 1 were used to prepare a 1.4 kb DNA fragment (P-rapA-Cm2) containing the chloramphenicol-resistant gene and a promoter region of the rapA gene by PCR. Separately, a primer set of comp_P-rapA R/BSpgsB atg FW (SEQ ID NO: 39) and pgsC FW and a genome DNA prepared from Bacillus subtilis 168 strain, as a template, were used to prepare a pgsB gene 1.2 kb DNA fragment (pgsB2) by PCR. Further, three fragments of the thus-prepared (pgsB2) and (P-rapA-Cm2) and the DNA fragment (pgsB-UP) in the Preparation Example 2, as templates, and a primer set of pgsC FW and pgsB RV were used to prepare a 3.5 kb DNA fragment by SOE-PCR.

Subsequently, the thus-prepared DNA fragment was used to transform Bacillus subtilis 168 strain by the competent cell method in the similar manner as in the Preparation Example 2 (hereinafter, the thus-obtained recombinant Bacillus subtilis is referred to as 1 cp-P_rapA/pgsBCAcm strain). A genome DNA sample was prepared from the obtained transformant, and this genome DNA sample, as a template, and a primer set of pgsC-R and BamHI_PrapA FW (SEQ ID NO: 40) were used to prepare a 2.2 kb DNA fragment (P_rapA/pgsBC) having a Bacillus subtilis rapA gene promoter upstream of the Bacillus subtilis pgsBC gene. Then, the thus-prepared DNA fragment was cloned into a plasmid vector in the same manner as in the Preparation Example 1, and the recombinant plasmid obtained was designated as recombinant vector for producing PGA pHY-P_rapA/pgsBC.

### Preparation Example 4

### Construction of Vector (4)

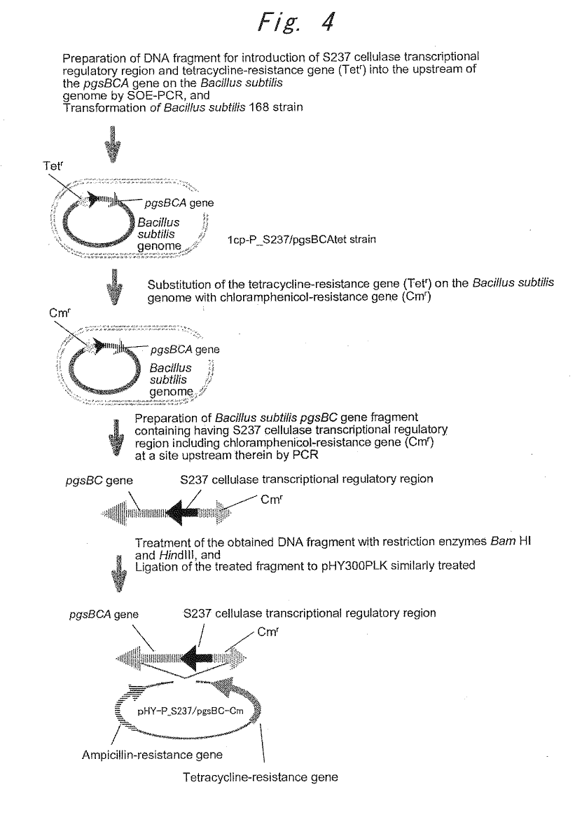

In this Preparation Example, a method of cloning genes relating to synthesis of PGA is described, in a similar manner to the Preparation Example 2 (see FIG. 4).

The pHY-P_S237/pgsBCA prepared in the Preparation Example 1, serving as a template, and a primer set of pgsC-R and tet4-1 (SEQ ID NO: 41) shown in Table 1 were used to prepare a 3.3 kb DNA fragment of pgsB gene (tet-P_B) containing a tetracycline resistance gene (Tet^{r}) on the plasmid, and an S237 cellulase promoter sequence in the upstream of the fragment. Separately, a chromosome DNA, serving as a template, prepared from Bacillus subtilis 168 strain and a primer set of tet4-1_comp/pgsB FW (SEQ ID NO: 42) and pgsB RV were used to prepare a 1.0 kb DNA fragment (pgsB-DW1) flanking the upstream of the pgsBCA gene on the Bacillus subtilis genome.

These fragments (tet-P_B) and (pgsB-DW1), as templates, and a primer set of pgsC-R and pgsB RV were used to bind the two fragments to each other by SOE-PCR method and to prepare a DNA fragment (tet-P_S237) for substitution of the promoter flanking the upstream of the pgsBCA gene operon of the Bacillus subtilis genome. Further, the obtained DNA fragment (tet-P_S237) was used to transform Bacillus subtilis 168 strain by the competent method, and a colony growing on a LBTc agar medium was separated as a transformant. The genome DNA sample was extracted from the transformant obtained, and PCR by using this sample as a template revealed that the transformant was a mutant Bacillus subtilis strain (hereinafter, referred to as 1 cp-P_S237/pgsBCAtet strain) having a S237 cellulase promoter flanking the upstream of the pgsBCA gene operon.

Then, a chromosome DNA prepared from Bacillus sp. KSM-S237 strain (FERM BP-7875), serving as a template, and a primer set of P_S237-RV and P_S237-F were used to prepare an approximately 0.6 kb DNA fragment of the S237 cellulase promoter region (PS237-Cm) by PCR method. Separately, the plasmid pC194, as a template, and a primer set of P_S237_comp/Cm FW (SEQ ID NO: 43) and CmRV (SEQ ID NO: 23) were used to prepare a 0.9 kb DNA fragment of the chloramphenicol-resistant gene (Cm-PS237).

Subsequently, a 3.2 kb DNA fragment was obtained by SOE-PCR by using a mixture of the thus-prepared DNA fragments (PS237-Cm) and (Cm-PS237) and the DNA fragment (BCA-UP) prepared in Preparation Example 6, as templates, and a primer set of P_S237-RV and pgsB RV. The thus-prepared DNA fragment was used to transform a mutant Bacillus subtilis strain (1 cp-P_S237/pgsBCAtet) by the competent cell method, and a colony growing on a LBCm agar medium was separated as a transformant.

Then, a chromosome DNA prepared from the thus-prepared transformant, serving as a template, and a primer set of pgsC-R and pC194 CmRV/HindIII RV (SEQ ID NO: 44) were used to prepare a 3.4 kb pgsBC gene fragment containing a chloramphenicol-resistant gene and having S237 cellulase promoter sequence upstream thereof. After purification and recovery, the DNA fragment obtained was treated with a restriction enzyme HindIII, and ligated with (bound to) a plasmid vector pHY300PLK previously treated with the same restriction enzyme by using a DNA Ligation kit Ver. 2. The above-described ligation sample was used to transform a Bacillus subtilis 168 strain by protoplast method, and a colony growing on a protoplast regeneration medium (DM3 medium) containing 5 ppm of chloramphenicol was collected as a transformant. The transformant obtained was grown once again on the LBCm agar medium, a plasmid was prepared from the microbe obtained by using High Pure Plasmid Isolation Kit, and the plasmid obtained was designated as a recombinant vector for producing PGA pHY-P_S237/pgsBC-Cm.

### Preparation Example 5

### Construction of Bacillus subtilis Mutant Strain (1)

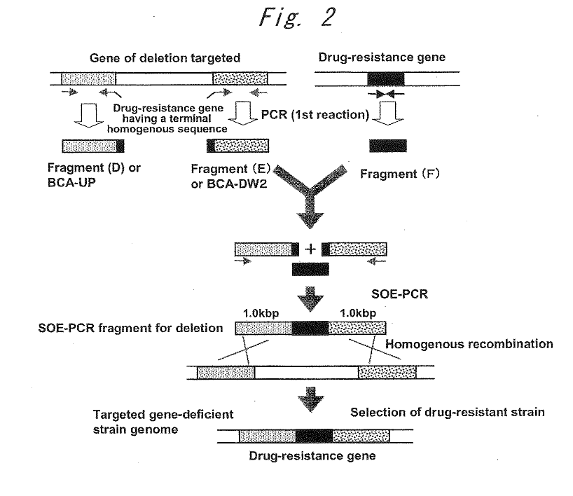

In this Preparation Example, a method of producing a Bacillus subtilis mutant strain for introduction of the vector obtained in the Preparation Example 1 is described (see FIG. 2).

First, a genome DNA prepared from Bacillus subtilis 168 strain, serving as a template, and any one of a primer set of ggt-F (SEQ ID NO: 18) and ggt/Cm-R (SEQ ID NO: 19) and a primer set of ggt/Cm-F (SEQ ID NO: 20) and ggt-R (SEQ ID NO: 21) shown in Table 1 were used to prepare a 1.0 kb DNA fragment (D) in contact with the upstream of the ggt gene on the genome and a 1.0 kb DNA fragment (E) in contact with the downstream of the ggt gene on the genome. Separately, the plasmid pC194, as a template, and a primer set of CmFW (SEQ ID NO: 22) and CmRV shown in Table 1 were used to prepare a 0.9 kb DNA fragment (F) containing a chloramphenicol-resistant gene.

Subsequently, the thus-prepared three fragments (D), (E) and (F) were mixed. Then, the thus-obtained template and a primer set of ggt-F and ggt-R were used to bind the three fragments each other in the order of (D)-(F)-(E) by SOE-PCR, to prepare a 2.9 kb DNA fragment for gene deletion. The thus-prepared DNA fragment was used to transform a Bacillus subtilis 168 strain by competent cell method, and a colony growing on a LBCm agar medium was separated as a transformant. Further, a genome DNA was extracted from the transformant obtained, and it was confirmed by the PCR using the DNA as a template that the transformant was a desirable Bacillus subtilis mutant strain (hereinafter, referred to as Aggt strain) in which the structural gene (ORF) of the ggt gene was replaced with the chloramphenicol-resistant gene (fragment F).

### Preparation Example 6

### Construction of Bacillus subtilis Mutant Strain (2)

In this Preparation Example, a method of producing a Bacillus subtilis mutant strain for introduction of the vector obtained in the Preparation Example 1 is described (see FIG. 2).

First, a genome DNA prepared from Bacillus subtilis 168 strain, serving as a template, and any one of a primer set of pgsA FW (SEQ ID NO: 45) and pgsA/Cm R (SEQ ID NO: 46) a primer set of pgsB/Cm F and pgsB RV shown in Table 1 were used to prepare a 1.0 kb DNA fragment (BCA-UP) in contact with the downstream of the pgsBCA gene on the genome and a 1.0 kb DNA fragment (BCA-DW2) in contact with the upstream of the pgsBCA gene on the genome.

Subsequently, the thus-prepared fragments (BCA-UP) and (BCA-DW2) and the DNA fragment (F) prepared in Preparation Example 5 were mixed to prepare a template. Then, the template and a primer set of pgsA FW and pgsB RV were used to bind the three fragments each other in the order of (BCA-UP)-(F)-(BCA-DW2) by SOE-PCR, to prepare a 2.9 kb DNA fragment for gene deletion. The thus-prepared DNA fragment for gene deletion was used to transform a Bacillus subtilis 168 strain by competent cell method, and a colony growing on a LBCm agar medium was separated as a transformant. Further, a genome DNA was extracted from the transformant obtained, and it was confirmed by the PCR using the DNA as a template that the transformant was a desirable Bacillus subtilis mutant strain (hereinafter, referred to as ΔBCA strain) in which the pgsBCA gene operon was replaced with the chloramphenicol-resistant gene (fragment F).

### Preparation Example 7

### Transformation by Introduction of Plasmid

In this Preparation Example, a method of transforming the Bacillus subtilis strain and the mutant strains thereof and other Bacillus microbes, as host cells.

Bacillus subtilis strains (Bacillus subtilis Marburg No. 168 strain, NCIMB11623 strain and IFO3215 strain), the Bacillus subtilis mutant strain prepared in Preparation Example 5 (Δggt strain), the Bacillus subtilis mutant strain prepared in Preparation Example 6 (ΔBCA strain), the 1_cp-P_S237/pgsBCAcm strain prepared in Preparation Example 4, the 1_cp-PrapA/pgsBCAcm strain prepared in Preparation Example 3, a Bacillus megaterium ATCC14581 strain, and a Bacillus megaterium WH320 strain were transformed respectively with the recombinant vectors for producing PGA (pHY-P_S237/pgsBCA, pHY-P_S237/pgsBC, pHY-P_spoVG/pgsBC and pHY-P_rapA/pgsBC) obtained in Preparation Examples 1, 2 and 3 and a control vector (pHY300PLK). The wild strains and the mutant strains were treated with lysozyme, to prepare 10⁵ to 10⁶ protoplasts. To thus-obtained protoplasts, 20 to 100 ng of the plasmid DNA were added for introduction of the plasmid. A colony growing on a DM3 protoplast regeneration medium (DM3 medium) containing 20 ppm tetracycline hydrochloride salt was collected as a desirable transformant Bacillus subtilis strain containing the plasmid.

The Bacillus licheniformis ATCC9945a strain was transformed by using the recombinant vector for producing PGA obtained in Preparation Example 4 (pHY-P_S237/pgsBC-Cm), and a colony growing on a DM3 protoplast regeneration medium (DM3 medium) containing 15 ppm of chloramphenicol was collected as a desired plasmid-incorporated transformant.

### Example 1

### Evaluation of Productivity (1)

The transformant prepared in Example 7, in which pHY-P_S237/pgsBCA or pHY-P_S237/pgsBC was introduced into the Bacillus subtilis 168 strain, was inoculated on a LBTc agar medium and cultured stationarily at 30° C. overnight. The Bacillus subtilis transformant growing on the LBTc agar medium was agitated and suspended in 0.8 to 1.6 mL of modified 2xL/Maltose medium (2.0% triptone, 1.0% yeast extract, 1.0% sodium chloride, 7.5% maltose, 7.5 ppm manganese sulfate tatra- or penta-hydrate, 15 ppm tetracycline hydrochloride salt) placed in a 2.0 mL small-capacity centrifuge tube previously sterilized, and the resulting supernatant after left still was used as seed culture liquid. The seed culture liquid was inoculated in a new modified 2xL/Maltose medium at 1% (v/v), and the medium was cultured in a Sakaguchi flask at 37° C. while shaken reciprocally at 120 rpm for 3 days (on TB-20R-3F, manufactured by Takasaki Scientific Instrument). After test culture, the amount of produced PGA was determined under the analytical conditions shown in the following measurement example and the evaluation results are summarized in Table 2.

TABLE 2

| | | Amount of produced PGA | | |
|---|---|---|---|---|
| | | Culture | Culture | |
| | | for | for | Culture for |
| Host cell | Vector introduced | one day | two days | three days |
| 168 strain | pHY-P_S237/pgsBC | 4.64 | 1.58 | 0.08 |
| | pHY-P_S237/pgsBCA | ND | ND | ND |

As shown in Table 2, the strain containing the introduced pgsBC gene had PGA productivity higher than that of the control strain containing the pgsBCA gene.

### Example 2

### Evaluation of Productivity (2)

The transformant prepared in Preparation Example 7, in which pHY-P_S237/pgsBCA or pHY-P_S237/pgsBC was introduced into the Bacillus subtilis 168 strain, was inoculated on a LBTc agar medium and cultured stationarily at 30° C. overnight. The Bacillus subtilis transformant was agitated and suspended in a modified 2xL/Maltose+MSG medium (2.0% triptone, 1.0% yeast extract, 1.0% sodium chloride, 7.5% maltose, 8.0% sodium glutamate monohydrate, 7.5 ppm manganese sulfate tatra- or penta-hydrate, and 15 ppm tetracycline hydrochloride salt), and the resulting supernatant after left still was used as seed culture liquid. The seed culture liquid was inoculated in a new modified 2xL/Maltose+MSG medium at 1% (v/v), and the medium was cultured in a Sakaguchi flask at 37° C. while shaken reciprocally at 120 rpm for 3 days (on TB-20R-3F, Takasaki Scientific Instrument). After test culture, the amount of produced PGA was determined under the analytical conditions shown in the following measurement example and the evaluation results are summarized in Table 3.

TABLE 3

| | | Amount of produced PGA | | |
|---|---|---|---|---|
| | | | Culture | Culture |
| | | Culture for | for | for |
| Host cell | Vector introduced | one day | two days | three days |
| 168 strain | pHY-P_S237/pgsBC | 30.8 | 28.1 | 30.8 |
| | pHY-P_S237/pgsBCA | 0.75 | 1.38 | 1.19 |

As shown in Table 3, the PGA productivity was increased particularly significantly when there was an excessive amount of glutamic acid contained in the medium. It was also found that the PGA productivity remained high even after culture for three days when there was an excessive amount of glutamic acid in the medium.

### Example 3

### Evaluation of Productivity (3)

The PGA productivity of each of the Bacillus subtilis 168 strain, and the transformant prepared in Preparation Example 5, in which pHY-P_S237/pgsBC was introduced into the Bacillus subtilis mutant strain (Δggt strain) prepared in Preparation Example 5, was evaluated in a similar manner to Example 1. After test culture, the amount of produced PGA was determined under the analytical conditions shown in the following measurement example and the evaluation results are summarized in Table 4.

TABLE 4

| | | Amount of produced PGA | | |
|---|---|---|---|---|
| | | | Culture | Culture |
| | | Culture for | for | for |
| Host cell | Vector introduced | one day | two days | three days |
| 168 strain | pHY-P_S237/pgsBC | 4.67 | 3.64 | 1.71 |
| Δggt strain | pHY-P_S237/pgsBC | 5.05 | 5.56 | 5.25 |

As shown in Table 4, the Δggt strain, when used as the host, was superior in PGA productivity to the wild strain Bacillus subtilis 168 strain, and the PGA productivity remained high even after culture for three days.

### Example 4

### Evaluation of Productivity (4)

The PGA productivity of each of the Bacillus subtilis 168 strain, and the transformant prepared in Preparation Example 7, in which pHY-P_S237/pgsBC was introduced into the Bacillus subtilis mutant strain (ΔBCA strain) prepared in Preparation Example 6, was evaluated in a similar manner to Example 1. After test culture, the amount of produced PGA was determined under the analytical conditions shown in the following measurement example and the evaluation results are summarized in Table 5.

TABLE 5

| | | Amount of produced PGA | | |
|---|---|---|---|---|
| | | Culture | Culture | |
| | | for | for | Culture for |
| Host cell | Vector introduced | one day | two days | three days |
| 168 strain | pHY-P_S237/pgsBC | 4.41 | 3.11 | ND |
| ΔBCA strain | pHY-P_S237/pgsBC | 4.76 | 3.38 | ND |
| ΔBCA strain | pHY300PLK | ND | ND | ND |

As shown in Table 5, not only the Bacillus subtilis 168 strain having pgsBCA gene on its chromosome but no ability of producing PGA, but also the ΔBCA strain deficient in the pgsBCA gene showed high PGA productivity.

### Example 5

### Evaluation of Productivity (5)

The PGA productivity of the transformant prepared in Example 7, in which pHY-P_S237/pgsBC, pHY-P_spoVG/pgsBC or pHY-P_rapA/pgsBC was introduced into Bacillus subtilis 168 strain, was evaluated in a similar manner to Example 2. After test culture, the amount of produced PGA was determined under the analytical conditions shown in the following measurement example and the evaluation results are summarized in Table 6.

TABLE 6

| | | Amount of produced PGA | | |
|---|---|---|---|---|
| | | Culture | Culture | |
| | | for | for | Culture for |
| Host cell | Vector introduced | one day | two days | three days |
| 168 strain | pHY-P_S237/pgsBC | | | 43.1 |
| | pHY-P_spoVG/pgsBC | | | 10.2 |
| | pHY-P_rapA/pgsBC | | | 67.3 |

As shown in Table 6, by using the rapA gene promoter and the spoVG gene derived from Bacillus subtilis, as well as the S237 cellulase promoter, the Bacillus subtilis transformant containing the pgsBC gene-containing recombinant vector for producing PGA showed high PGA productivity.

### Example 6

### Evaluation of Productivity (6)

The PGA productivity of each of the transformants prepared in Preparation Example 7, in which pHY-P_S237/pgsBC was introduced into the Bacillus subtilis strain NCIMB11623 and IFO3215, was evaluated in a similar manner to Example 2. After test culture, the amount of produced PGA was determined under the analytical conditions shown in the following measurement example and the evaluation results are summarized in Table 7.

TABLE 7

| | | Amount of produced PGA | | |
|---|---|---|---|---|
| | | Culture | Culture | Culture |
| | | for | for | for |
| Host cell | Vector introduced | one day | two days | three days |
| 168 strain | pHY-P_S237/pgsBC | | | 35.5 |
| NCIMB11623 | pHY-P_S237/pgsBC | | | 25.0 |
| strain | pHY300PLK | | | ND |
| IFO3215 | pHY-P_S237/pgsBC | | | 11.5 |
| strain | pHY300PLK | | | ND |

As shown in Table 7, introduction of the recombinant vector for producing PGA containing only the pgsBC gene to Bacillus subtilis 168 strain as well as other Bacillus subtilis species was effective in providing PGA productivity.

### Example 7

### Evaluation of Productivity (7)

The PGA productivity of the transformant prepared in Example 7, in which pHY-P_S237/pgsBC-Cm was introduced into Bacillus licheniformis ATCC9945a strain, was evaluated in a similar manner to Example 2 (in this case, as the antibiotic to be used, 15-ppm tetracycline was changed to 5-ppm chloramphenicol). After test culture, the amount of produced PGA was determined under the analytical conditions shown in the following measurement example and the evaluation results are summarized in Table 8.

TABLE 8

| | | Amount of produced PGA | | |
|---|---|---|---|---|
| | | Culture | Culture | Culture |
| | | for | for two | for |
| Host cell | Vector introduced | one day | days | three days |
| ATCC9945a | No plasmid introduced | | | 0.96 |
| | pHY-P_S237/pgsBC-Cm | | | 3.00 |

As shown in Table 8, introduction of recombinant vector for producing PGA containing the pgsBC gene to, as well as Bacillus subtilis, other Bacillus microbes was effective in increasing the PGA productivity.

### Example 8

### Evaluation of Productivity (8)

The PGA productivity of each of the transformant prepared in Preparation Example 7, in which pHY-P_spoVG/pgsBC was introduced into Bacillus megaterium ATCC14581 strain, and the transformant prepared in Preparation Example 7, in which pHY-P_S237/pgsBC was introduced into Bacillus megaterium WH320 strain, was evaluated in a similar manner to Example 2. After test culture, the amount of produced PGA was determined under the analytical conditions shown in the following measurement example and the evaluation results are summarized in Table 9.

TABLE 9

| | | Amount of produced PGA | | |
|---|---|---|---|---|
| | | Culture | Culture | Culture |
| | | for | for | for |
| Host cell | Vector introduced | one day | two days | three days |
| ATCC14581 | pHY300PLK | | | 0.05 |
| strain | pHY-P_spoVG/pgsBC | | | 0.55 |
| WH320 | pHY300PLK | | | 5.05 |
| strain | pHY-P_S237/pgsBC | | | 6.98 |

As shown in Table 9, introduction of recombinant vector for producing PGA containing the pgsBC gene to, as well as Bacillus subtilis, other Bacillus microbes was effective in increasing the PGA productivity.

### Example 9

### Evaluation of Productivity (9) (Molecular Weight Evaluation)

The PGA productivity employing each of the transformant prepared in Preparation Example 7, in which pHY-P_S237/pgsBC was introduced into the 1cp-P_rapA/pgsBCAcm strain and the 1 cp-P_S237/pgsBCAcm strain prepared in Preparation Examples 3 and 4, was evaluated in a similar manner to Example 2. After test culture, the amount of PGA produced and the molecular weight thereof were determined under the analytical conditions shown in the following measurement example and the evaluation results are summarized in Tables 10 and 11.

TABLE 10

| | | Amount of produced PGA | | |
|---|---|---|---|---|
| | | Culture | Culture | Culture |
| | | for | for | for |
| Host cell | Vector introduced | one day | two days | three days |
| 1cp-P_S237/ | pHY300PLK | | | 28.9 |
| pgsBCAcm | pHY-P_S237/pgsBC | | | 39.4 |
| 1cp-P_rapA/ | pHY300PLK | | | 3.2 |
| pgsBCAcm | pHY-P_S237/pgsBC | | | 33.2 |

As shown in Table 10, introduction of a recombinant vector for producing PGA having only the pgsBC gene into the Bacillus subtilis mutant capable of producing PGA was found to increase PGA productivity, compared to the strain without introduction of the gene.

TABLE 11

| | Vector | Molecular weight of PGA | |
|---|---|---|---|
| Host cell | introduced | Culture for one day | Culture for three days |
| 1cp-P_S237/ | pHY300PLK | 7,000,000 to 8,500,000 | 6,300,000 to 7,600,000 |
| pgsBCAcm | pHY-P_S237/ | 7,500,000 to 10,400,000 | 6,200,000 to 9,100,000 |
| | pgsBC | | |
| 1cp-P_rapA/ | pHY300PLK | 340,000 to 860,000 | 350,000 to 830,000 |
| pgsBCAcm | pHY-P_S237/ | 6,900,000 to 12,400,000 | 5,900,000 to 8,100,000 |
| | pgsBC | | |

As shown in Table 11, introduction of a recombinant vector for producing PGA having only the pgsBC gene into the Bacillus subtilis mutant capable of producing PGA was found to increase the molecular weight of the PGA produced, compared to the strain without introduction of the gene.

### Measurement Example

### Method of Quantitatively Determining Amount of PGA Produced and Measuring the Molecular Weight of PGA Produced

Each of the culture samples after test culture shown in Examples 1 to 9 was centrifuged at room temperature at 14,800 rpm for 30 minutes (trade name: himac CF15RX, Hitachi Koki), and the amount of the PGA produced by using the recombinant microorganisms in the culture supernatant obtained after centrifugation was determined. PGA in the supernatant sample was analyzed according to the method by Yamaguchi et al. (described above). Specifically. PGA production by using the recombinant microorganisms was confirmed by subjecting the sample to agarose gel electrophoresis, staining the gel with methylene blue, and observing the presence of stained substances derived from PGA. The sample containing PGA produced by the recombinant microorganisms thus detected was analyzed by HPLC using TSKGel G4000PWXL and TSKGel G6000PWXL gel filtration columns (trade name, Tosoh Corporation). As for the analytical condition, the eluate used was 0.1 M sodium sulfate, the flow rate was 1.0 mL/minute, the column temperature was 50° C., and the UV detection wavelength was 210 nm. In determination of the concentration, a PGA having a molecular weight of 800 k (Meiji Food Materia) was used for preparation of a calibration curve. For determination of the molecular weight, various poly-glutamic acid samples having different molecular weights [Wako Pure Chemical Industries (162-21411, 162-21401), SIGMA-ALDRICH (P-4886, P-4761), Meiji Food Materia (molecular weight: 880 k)], which were previously determined by using pullulan (Shodex STANDRD P-82, trade name, Showa Denko K.K.), were used as standards. The unit of the values showing the PGA-productivity in the Tables showing the evaluation results, is (g/L\. The notation “ND” in the Tables showing the evaluation results, means that the amount of PGA produced did not attain the detection limit.

### Example 10

### Evaluation of Productivity (10) (Measurement of Optical Purity of PGA Composition)

The culture solution obtained from the Bacillus subtilis 168 strain transformant having pHY-P_S237/pgsBCA introduced in Example 2, and the culture solution obtained the transformant of the Bacillus subtilis mutant strain (ΔBCA strain) having pHY-P_S237/pgsBC introduced in Example 4 were used as samples for measurement of optical purity. An equivalent amount of ethanol was added to the supernatant of the culture, and the precipitates generated were collected by centrifugation. Subsequently, the collected precipitates were subjected to dryness, and the resultant was encapsulated with 6N hydrochloric acid under nitrogen and heat-treated at 115° C. to 120° C. for 24 hours. After the heat treatment, hydrochloric acid and water were removed by distillation under nitrogen gas flow, to give a hydrolysate sample. Subsequently, the amino acid composition and the L-glutamic acid content in the hydrolysate sample were analyzed quantitatively by using a full automatic amino acid analyzer L-8500 (trade name, Hitachi Instrument Service) and L-glutamic acid measurement kit (Yamasa Corp). The analysis by using the full automatic amino acid spectrometer gave the total content of the optically active isomers (D- and L-isomers) quantitatively, and the D-isomer content was calculated by subtracting the quantitative content obtained by the L-isomer measurement kit from the total content. The PGA prepared by the Bacillus sp. KSM-366 strain (FERM BP-6262) was used as the PGA sample produced by wild-type Bacillus microbe. For evaluation of the racemization rate of the amino acid during the hydrolytic reaction. D- and L-glutamic acids (Wako Pure Chemical Industries) were used as standard samples. The results are summarized in Table 12.

TABLE 12

| | Glutamic acid | |
|---|---|---|
| | composition | |
| Sample | D-isomer | L-isomer |
| Supernatant of Culture of 168 strain | 75 | 25 |
| containing pHY-P_S237/pgsBCA introduced | | |
| Supernatant of Culture of ΔBCA strain | 6 | 94 |
| containing pHY-P_S237/pgsBC introduced | | |
| PGA produced by Bacillus sp. KSM-366 | 79 | 21 |
| strain L-Glutamic acid | 7 | 93 |
| (reagent: for evaluation of racemization rate) | | |
| D-Glutamic acid | 91 | 9 |
| (reagent: for evaluation of racemization rate) | | |

As shown in Table 12, the recombinant Bacillus subtilis containing the introduced recombinant vector for producing PGA having only the pgsBC gene produced a glutamic acid at a favorably high optical purity of L-glutamic acid content of 90% or more. The racemization rate of an amino acid under the hydrolytic condition separately analyzed was approximately 10%. From these facts, it was indicated that the optical purity of the PGA obtained by the recombinant Bacillus subtilis containing only the pgsBC gene introduced was 80% to 100%.

### Reference Example

In a similar manner to Preparation Example 1, a chromosome DNA, serving as a template, prepared from Bacillus sp. KSM-366 stain (FERM BP-6262) and a primer set of pgsB-F and pgsB-R (SEQ ID NO: 15) shown in Table 1 were used to prepare a 1.2 kb DNA fragment (G) of the pgsB gene. Separately, the DNA fragment (C) of the cellulase gene promoter region derived from KSM-S237 strain, serving as a template, and a primer set of P_S237-FW and pgsB-R were used to prepare a composite 1.8 kb DNA fragment (GC) of fragments (G) and (C) by SOE-PCR method. A recombinant plasmid containing the DNA fragment (GC) inserted between the BamHI and HindIII restriction enzyme recognition sites of plasmid vector pHY300PLK (Takara) was prepared then by an experimental method similar to that shown in Preparation Example 1, and was designated as recombinant vector for producing PGA pHY-P_S237/pgsB.

Using pHY300PLK having no PGA production gene introduced and the plasmid expressing only the pgsB gene (pHY-P_S237/pgsB) described above, the PGA productivity was determined by the methods described in Example 2 and 3. As a result, in both cases, it was confirmed that no PGA was produced.

### INDUSTRIAL APPLICABILITY

The recombinant microorganism according to the present invention has ability of producing poly-γ-glutamic acid. Thus, the recombinant microorganism according to the present invention can be used preferably in production of poly-γ-glutamic acid. It is also possible to produce a poly-γ-glutamic acid having desired molecular weight and structure, by employing the recombinant microorganism according to the present invention. Thus, the recombinant microorganism according to the present invention can be used preferably for improvement in productivity of the poly-γ-glutamic acid, and adjusting molecular weight and optical purity of the poly-γ-glutamic acid.

Having described our invention as related to the present embodiments, it is our intention that the invention not be limited by any of the details of the description, unless otherwise specified, but rather be construed broadly within its spirit and scope as set out in the accompanying claims.

This application claims priority on Patent Application No. 2007-244023 filed in Japan on Sep. 20, 2007, of which is entirely herein incorporated by reference.

### [Sequence Listing]

P98262TH.ST25.txt

What is claimed is:

1. A recombinant microorganism having the ability to produce poly-γ-glutamic acid,
wherein, the recombinant microorganism is prepared by introducing a Bacillus subtilis pgsB gene or a gene functionally equivalent thereto, and a Bacillus subtilis pgsC gene or a gene functionally equivalent thereto, among a group of genes relating to synthesis of poly-γ-glutamic acid, into a host microorganism, and
wherein no Bacillus subtilis pgsA gene or a gene functionally equivalent thereto is introduced into the host microorganism.

2. The recombinant microorganism according to claim 1, wherein the pgsB gene is a gene coding the following protein (a) or (b):
(a) a protein having the amino acid sequence set forth in SEQ ID NO: 2; or
(b) a protein having an amino acid sequence, in which one or more amino acids are deleted, substituted, added or inserted in the amino acid sequence set forth in SEQ ID NO: 2, and having amido-ligase activity.

3. The recombinant microorganism according to claim 1, wherein the pgsC gene is a gene coding the following protein (c) or (d):
(c) a protein having the amino acid sequence set forth in SEQ ID NO: 4; or
(d) a protein having an amino acid sequence, in which one or more amino acids are deleted, substituted, added or inserted in the amino acid sequence set forth in SEQ ID NO: 4, and having a function of producing poly-γ-glutamic acid in the presence of the PgsB protein coded by the pgsB gene.

4. The recombinant microorganism according to claim 1, wherein the pgsB gene or the gene functionally equivalent thereto, and the pgsC gene or the gene functionally equivalent thereto, are incorporated in a plasmid self-replicable in a cell of the host microorganism.

5. The recombinant microorganism according to claim 1, wherein the pgsB gene or the gene functionally equivalent thereto, and the pgsC gene or the gene functionally equivalent thereto, have a transcription initiation regulatory region and/or a translation initiation regulatory region each functioning in the microorganism at a particular site upstream of the genes.

6. The recombinant microorganism according to claim 1, wherein the host microorganism is a Bacillus microbe.

7. The recombinant microorganism according to claim 6, wherein the Bacillus microbe is Bacillus subtilis.

8. A method of producing a poly-γ-glutamic acid, comprising the steps of:
culturing the recombinant microorganism according to claim 1, and
collecting the poly-γ-glutamic acid that is produced.

9. A method of improving a productivity in poly-γ-glutamic acid production, which comprises employing a recombinant microorganism, which is obtained by introducing a Bacillus subtilis pgsB gene or a gene functionally equivalent thereto, and a Bacillus subtilis pgsC gene or a gene functionally equivalent thereto, among a group of genes relating to the synthesis of poly-γ-glutamic acid, into a host microorganism.

10. A method of improving a productivity in poly-γ-glutamic acid production, which comprises employing a recombinant microorganism, which is obtained by introducing a Bacillus subtilis pgsB gene or a gene functionally equivalent thereto, and a Bacillus subtilis pgsC gene or a gene functionally equivalent thereto among a group of genes relating to synthesis of poly-γ-glutamic acid, into a host microorganism, wherein the recombinant microorganism is the recombinant microorganism according to claim 1.

11. A method of adjusting the molecular weight of poly-γ-glutamic acid, which comprises employing a recombinant microorganism, which is obtained by introducing a Bacillus subtilis pgsB gene or a gene functionally equivalent thereto, and a Bacillus subtilis pgsC gene or a gene functionally equivalent thereto, among a group of genes relating to the synthesis of poly-γ-glutamic acid, into a host microorganism.

12. A method of adjusting the molecular weight of poly-γ-glutamic acid, which comprises employing a recombinant microorganism, which is obtained by introducing a Bacillus subtilis pgsB gene or a gene functionally equivalent thereto, and a Bacillus subtilis pgsC gene or a gene functionally equivalent thereto, among a group of genes relating to the synthesis of poly-γ-glutamic acid, into a host microorganism, wherein the recombinant microorganism is the recombinant microorganism according to claim 1.

13. A method of producing a high-molecular-weight poly-γ-glutamic acid, which comprises employing the method of adjusting the molecular weight of poly-γ-glutamic acid according to claim 11.

14. A method of adjusting the optical purity of poly-γ-glutamic acid, wherein the L-isomer ratio of the poly-γ-glutamic acid produced is controlled by employing a recombinant microorganism, which is obtained by introducing a Bacillus subtilis pgsB gene or a gene functionally equivalent thereto, and a Bacillus subtilis pgsC gene or a gene functionally equivalent thereto, among a group of genes relating to synthesis of poly-γ-glutamic acid, into a host microorganism.

15. A method of adjusting the optical purity of poly-γ-glutamic acid,
wherein the L-isomer ratio of the poly-γ-glutamic acid produced is controlled by employing a recombinant microorganism, which is obtained by introducing a Bacillus subtilis pgsB gene or a gene functionally equivalent thereto, and a Bacillus subtilis pgsC gene or a gene functionally equivalent thereto, among a group of genes relating to the synthesis of poly-γ-glutamic acid, into a host microorganism, and
wherein the recombinant microorganism is the recombinant microorganism according to claim 1.

16. A method of producing poly-γ-glutamic acid having a high L-isomer ratio, which comprises employing the method of adjusting the optical purity of poly-γ-glutamic acid according to claim 14.

17. Use of the recombinant microorganism according to claim 1.
